(19) United States
(12) Patent Application Publication
(10) Pub. No.: US 2023/0000816 A1
(43) Pub. Date: Jan. 5, 2023

(54) SODIUM-GLUCOSE LINKED TRANSPORTER INHIBITORS FOR THE MANAGEMENT OF CHRONIC KIDNEY DISEASE, HYPERTENSION, AND HEART FAILURE IN COMPANION ANIMALS

(71) Applicant: INCREVET, INC., Boston, MA (US)

(72) Inventors: Michael HADD, San Jose, CA (US); Brian SEED, Boston, MA (US); Thomas John DUPREE, Cumberland, ME (US); Jordan MECHANIC, San Jose, CA (US)

(21) Appl. No.: 17/775,029

(22) PCT Filed: Nov. 6, 2020
(86) PCT No.: PCT/US2020/059358

§ 371 Date: May 6, 2022

Related U.S. Application Data
(60) Provisional application No. 62/932,395, filed on Nov. 7, 2019.

(51) Int. Cl. A61K 31/351; A61K 31/4965; A61P 13/12; A61P 9/04; A61P 3/10; A61P 9/12

(52) U.S. Cl. CPC A61K 31/351; A61K 31/4965; A61P 13/12; A61P 9/04; A61P 3/10; A61P 9/12

(57) ABSTRACT

Provided herein are methods of treating heart failure, chronic kidney disease (CKD) or hypertension, in a companion animal, comprising administering to a companion animal in need thereof a therapeutically effective amount of a compound that inhibits a sodium-dependent glucose transporter (SGLT) or a prodrug thereof. In some embodiments, the compound that inhibits an SGLT is bexagliflozin.

### CROSS-REFERENCES TO RELATED APPLICATIONS

This application claims the benefit of priority under 35 U.S.C § 119(e) to U.S. Provisional Application Ser. No. 62/932,395 filed Nov. 7, 2019, which is incorporated herein by reference in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND

Heart failure, chronic kidney disease (CKD), and hypertension are well studied and discussed diseases that reduce quality of life and shorten life expectancy in human populations. Less studied are the effects of these diseases on the companion animal population. Despite the lesser attention, heart failure, chronic kidney disease, and hypertension are also prevalent in companion animals and contribute to or cause a significant number of deaths in these populations.

As such, there is a need in the art for identifying therapeutic options for treating heart failure, chronic kidney disease (CKD), and hypertension in companion animals. The present disclosure addresses this need and provides related advantages as well.

### BRIEF SUMMARY

In some aspects, provided herein are methods for the treatment of heart failure in a companion animal, comprising administering to the companion animal in need thereof a therapeutically effective amount of a compound that inhibits a sodium-dependent glucose transporter (SGLT) or a prodrug thereof.

In additional aspects, provided herein are methods for the treatment of chronic kidney disease (CKD) in a companion animal, comprising administering to the companion animal in need thereof a therapeutically effective amount of a compound that inhibits a sodium-dependent glucose transporter (SGLT) or a prodrug thereof.

In further aspects, provided herein are methods for the treatment of hypertension in a companion animal, comprising administering to the companion animal in need thereof a therapeutically effective amount of a compound that inhibits a sodium-dependent glucose transporter (SGLT) or a prodrug thereof.

In some embodiments, the compound that inhibits an SGLT or a prodrug thereof is a compound described herein.

In some embodiments the companion animal is a canine.

In some embodiments the companion animal is a feline.

### BRIEF DESCRIPTION OF THE DRAWINGS

FIG. 1 displays the plasma volume after 7-day exposure to pioglitazone. 10 rats per group were exposed to pioglitazone, approximately 25 mg kg⁻¹ day⁻¹, admixed in the diet. ⁺, plasma volume increased to a statistically significant (p<0.001) degree for rats in the control (con) and amiloride (ami) groups, but not in the bexagliflozin (bex) group. *, the volume in the bexagliflozin group after 7 days differed from that of the control group to a statistically significant (p<0.01) degree. Bars represent standard deviations.

FIG. 2 displays the change in plasma volume from day 1 to day 7. The change in plasma volume from day 1 to day 7 is plotted, with standard deviations. *, the change in volume in the bexagliflozin group (hex) differed from that of the control group (con) to a significant (p<0.05) degree but that of the amiloride group (ami) did not.

FIG. 3 displays the calculated pioglitazone dose. The pioglitazone dose was estimated by dividing the amount consumed in food by the animal weights on days 1 and 7. Rats in the bexagliflozin group (hex) consumed more food and gained less weight than rats in the control (con) or amiloride (ami) groups, leading to larger predicted pioglitazone doses. Intergroup differences did not reach significance.

FIG. 4 shows the bexagliflozin plasma concentration in dogs as a function of time. Four dogs per group were dosed with bexagliflozin at the indicated number of mg kg⁻¹ and venous blood specimens were drawn at intervals thereafter.

FIG. 5 displays the glucosuria in dogs produced by bexagliflozin. The 24 h urinary glucose excretion produced by beagle dogs administered bexagliflozin in the form of a 1:2 bexagliflozin:proline co-crystal is plotted as a function of bexagliflozin dose in mg kg⁻¹. Doses varied from 0.02 to 2.0 mg kg⁻¹. The solid line represents the equation shown in the inset, which is the result of a fit of the data by non-linear minimization.

FIG. 6 displays the creatinine measurements from diabetic cats managed with bexagliflozin.

FIG. 7 displays the BUN measurements from diabetic cats managed with bexagliflozin.

FIG. 8 displays the SDMA measurements from diabetic cats managed with bexagliflozin.

FIG. 9 displays the veterinarian mean assessments of musculature for diabetic cats managed with bexagliflozin.

FIG. 10 displays the owner mean assessments of musculature for diabetic cats managed with bexagliflozin.

FIG. 11 displays the owner mean assessments of activity of diabetic cats managed with bexagliflozin.

FIG. 12 displays the owner mean assessments of leaping ability of diabetic cats managed with bexagliflozin.

FIG. 13 displays the least squares mean weight of cats by visit with 95% confidence intervals.

### DETAILED DESCRIPTION

### I. General

This application discloses the utility of sodium-glucose linked transporter (SGLT) inhibitors for the management hypertension, renal disease (e.g., chronic kidney disease) or heart failure in companion animals (in particular, felines and canines).

### Sodium-Glucose Linked Transporters (SGLTs)

SGLT proteins belong to a phylogenetically related collection of transporters genetically encoded by genes of the solute-linked carrier 5A (SLC5A) family. SGLT1 and SGLT2 are related transporters, encoded by SLC5A1 and SLC5A2, respectively, in humans, that are responsible for the reuptake of glucose from the renal filtrate. SGLT1 also plays a substantial role in the absorption of glucose from the intestine. Both transporters rely on the transmembrane electrochemical gradient for sodium to actively transport glucose across the cell membrane and into the cells of the renal proximal tubule. SGLT2, which cotransports one molecule of glucose and one sodium ion, is predominantly located in the initial portion of the proximal tubule, known as the pars convoluta, or convoluted portion of the proximal tubule, whereas SGLT1, which cotransports one molecule of glucose and two sodium ions, is predominantly located downstream of SGLT2, in the pars recta, or straight portion of the proximal tubule. Under ordinary circumstances SGLT2 takes up more than 90% of the glucose and SGLT1 takes up the remainder. SGLT2 has a weaker concentrative power than SGLT1, but accomplishes uptake by an energetically more favorable mechanism. Ultimately the Na⁺ ions that are cotransported into the cell must be expelled by an energy-requiring (ATP-consuming) process.

Loss of SGLT2 function as a result of genetic mutation results in glucosuria but loss of function of SGLT1 does not. Nonetheless, when SGLT2 is inhibited, SGLT1 can partially compensate. Highly specific SGLT2 inhibitors developed for the treatment of type 2 diabetes mellitus (T2DM) in humans typically produce a maximum urinary glucose excretion of approximately 80 g per day in healthy volunteers, approximately half of the expected glomerular flux. The remainder is believed to be reabsorbed by SGLT1.

The diversion of glucose from the bloodstream to urine represents an attractive mechanism for the treatment of T2DM because individuals lacking SGLT2 are euglycemic and typically healthy, indicating that complete loss of the transporter is well-tolerated. In addition, T2DM is very frequently triggered by excess weight and the dissipation of plasma glucose in urine instead of assimilation into fat, thereby exacerbating the root cause of the disease, is seen as an appealing alternative to the use of insulin and insulin sensitizers and secretagogues, which tend to cause weight gain as a result of the action of insulin on adipocytes. However, one disadvantage of SGLT2 inhibition is that the mechanism is dependent on renal function, and as the latter wanes, so does the therapeutic benefit. This has been empirically demonstrated by measurement of the urinary glucose excretion by study populations with T2DM and varying degrees of renal compromise. No SGLT2 inhibitors have been approved to date for the treatment of T2DM in humans with advanced moderate CKD (stage 3b, corresponding to an eGFR of 30-44 mL min⁻¹ per 1.73 m²).

### Chronic Kidney Disease (CKD)

The kidneys are twin organs responsible for the excretion of waste products that develop in the course of resting metabolism in a large number of species. Except in rare conditions, such as hibernation, the usual consequences of ongoing metabolism result in the accumulation of decomposition products of nucleic acids, proteins and vitamins that if not discharged from the body would ultimately impair its proper functioning. In addition, as the principal organ for the elimination of dietary and environmental toxins, the kidney has an especially important role to play in the survival of terrestrial vertebrates. By far the largest fraction of ingested toxins in the diets of herbivores and omnivores is contributed by plant secondary metabolism, which in aggregate creates an extraordinary variety of toxic compounds to protect plants from consumption.

The kidney functions by a default release system, in which all of the contents of the plasma below approximately 50 kDa in mass are excreted, and then only the plasma contents of interest are reabsorbed. In this way toxins, and in general all soluble foreign compounds introduced into the bloodstream, are excreted by default. The process of selective reabsorption is quite expensive metabolically, and it has been estimated that approximately 25% of the resting energy requirements of humans are devoted to the process of renal filtration and reabsorption. The task is substantial because all of the water, electrolytes, vitamins and most metabolites must be reabsorbed.

Anatomically, the kidney is a bundle of individual tubes that follow complex paths from their origins in the glomeruli, the ball-like structures through which blood flows and plasma is released, to the ureter, the final collecting structure into which all of the individual tubes eventually fuse. Each individual structural unit of a glomerulus and tubule is called a nephron. A feedback loop is created within each nephron to control flow and is made possible by an anatomical organization in which a distal segment of each nephron loops back to come into contact with the glomerulus from which it emanated. A sensing mechanism allows the blood pressure across the glomerulus to increase or decrease as the filtrate flow in the tubule is sensed to be too low or too high, respectively. This control mechanism is referred to as tubuloglomerular feedback, and takes place in the juxtaglomerular apparatus, which as its name suggests, is a structure adjacent to the glomerulus in which the tubule and the blood supply (entering afferent arteriole and exiting efferent arteriole) are brought into apposition.

Loss of renal function typically results in mortality if not managed therapeutically. In humans, loss of renal function is most frequently due to chronic kidney disease (CKD), an indolent progressive condition that results in end stage renal disease (ESRD), a state that must be treated by dialysis or renal transplantation. Damage to the kidneys as a result of diabetes or hypertension are the two most common contributing factors for the progression of CKD in humans.

CKD is also a common condition afflicting canines and felines. According to a publication of the International Renal Interest Society (IRIS), CKD has a prevalence in dogs of between 0.5 and 1% and a prevalence in cats of between 1 and 3% (http://www.iris-kidney.com/education/risk_factors.html). The incidence of CKD increases with age in both species, and CKD is a common cause of feline mortality.

CKD is a progressive disorder and pharmacological or dietary interventions generally aim to slow the progression of the disease or its secondary complications. Damage to the kidneys is usually irreversible and there are no presently approved treatments to restore function to failing kidneys. As kidney function declines, the loss of individual nephrons manifests as a dwindling rate of filtration (a reduction in the glomerular filtration rate or GFR) and damage to the glomeruli results in a loss of integrity of the glomerular barrier function that allows the kidney to retain proteins of molecular mass greater than 50 kDa. As a result, proteinuria, the presence of abnormally large amounts of protein in urine, becomes apparent. Reductions in GFR have the effect of decreasing the rate at which toxins exit the body, and results in a generalized toxemia, or elevation of toxins in the blood and azotemia, the pathological accumulation of nitrogenous waste products, typically in the form of urea, in the blood.

Although it is possible to measure the GFR directly by infusion of appropriate tracer compounds into the bloodstream and measuring their disappearance as a function of time, in routine clinical practice, surrogate measures of the filtration rate are commonly employed. The most widely used of these in both human and animal medical practice is the measurement of the serum creatinine concentration. Creatinine is produced endogenously by muscle at a constant rate per gram of muscle at rest, with levels elevated somewhat by exertion. It is excreted by the kidney predominantly by glomerular filtration (in humans about 15% is excreted by an active transport process in the proximal tubule) and is not reabsorbed. Creatinine levels change little with time in most individuals, and as the GFR declines, the creatinine concentration in the blood increases.

Most measurements are made of serum creatinine. Serum is the liquid phase remaining after coagulation of blood and consists of plasma (the liquid phase of blood), and platelet proteins released upon coagulation. The relationship between the serum creatinine and the GFR is calculated by one or more equations, such as the Cockcroft-Gault equation or the Modification of Diet in Renal Disease (MDRD) equation for humans, that result in a derived quantity referred to as the estimated glomerular filtration rate, or eGFR. eGFR in humans is usually expressed in the form appropriate to a standard human with a surface area of 1.73 m², so that eGFR is expressed in mL min⁻¹ per 1.73 m². In veterinary practice, normalization by body mass instead of surface area is generally performed or the creatinine concentration per se is taken to be an indicator of renal health. The IRIS staging system for classifying CKD severity divides cats and dogs into four stages from healthy to severely diseased (http://www.iris-kidney.com/pdf/IRIS_Staging_of_CKD_modified_2019.pdf). The serum creatinine concentrations (often referred to interchangeably as blood creatinine in veterinary literature) for the four stages in dogs, expressed as mg of creatinine per dL of serum (mg dL⁻¹), are <1.4, 1.4-2.8, 2.9-5.0 and >5.0 mg dL⁻¹. Expressed as μmol per liter, these are <125, 125-250, 251-440 and >440 μmol L⁻¹. For cats the four stages are <1.6, 1.6-2.8, 2.9-5.0 and >5.0 mg dL⁻¹, or <140, 140-250, 251-440 and >440 μmol L⁻¹. Serum creatinine can be influenced by diet to some extent, as it and its precursor compound, creatine, can be found in meat. Hence a shift in diet away from carbohydrates, for example, would typically produce an increase in serum creatinine.

Another surrogate measure of renal function is the concentration of blood urea nitrogen (BUN, actually measured as serum or plasma urea nitrogen), which quantifies the amount of nitrogen in serum/plasma in the form of urea. Urea is formed by the liver as a detoxification of ammonia, produced predominantly from amino acid catabolism. Urea undergoes glomerular filtration but is also reabsorbed to a certain extent, with a net excretion of 30-50% of the filtrate urea. As with creatinine, the BUN increases with decreasing glomerular filtration.

Recently, a new analyte for the detection and estimation of renal disease, symmetrical dimethylarginine (SDMA), has become more frequently included in clinical chemistry panels for the assessment of companion animal health (http://www.iris-kidney.com/pdf/003-5559.001-iris-website-symmetric-dimethylarginine-pdf 220116-final.pdf). Methylation of arginine in vivo typically occurs on protein arginine residues and is carried out by specific protein arginine methylases, and reversed by specific protein arginine demethylases. SDMA bears one methyl group on each of the arginine guanidino group primary amine moieties, whereas asymmetrical dimethylarginine bears both methyl groups on one of the guanidino group primary amines. SDMA is produced by specific protein methylases and released upon protein degradation. It undergoes glomerular filtration but is not reabsorbed by the kidney and thus can serve as an indicator of glomerular filtration rate. It has the theoretical advantage of being little affected by diet. It has been proposed to be more sensitive than creatinine for the early detection of CKD, and because its production is not limited to muscle, changes in body composition are less likely to affect SDMA than to affect creatinine.

IRIS staging of CKD informed by SDMA is modified by adding SDMA criteria to the creatinine-based classification system. If a dog is considered healthy by creatinine (stage 1, <1.4 mg dL⁻¹) but has an SDMA that is persistently >18 μg dL⁻¹, the dog should be advanced to IRIS stage 2 and managed as such. Similarly, if the dog is at IRIS stage 2 and has an SDMA that is persistently >35 μg dL⁻¹, the dog should be advanced to stage 3 and so managed, and if the dog is at stage 3 and has an SDMA that is persistently >54 μg dL⁻¹, the dog should be advanced to stage 4 and so managed. For cats the threshold values for advancement in stage are 18, 25 and 38 μg dL⁻¹ of SDMA.

As mentioned above, proteinuria is a manifestation of renal disease and the degree of proteinuria is a measure of disease severity. The most abundant protein in plasma is albumin, and the degree of proteinuria in humans is often presented as the urinary albumin to creatinine ratio or UACr. Albuminuria stage A1 is described by a UACr of <30 mg g⁻¹, stage A2 by 30≤UACr≤300 mg g⁻¹ and stage A3 by UACr>300 mg g⁻¹. These are sometimes described in units of mg mmol⁻¹, for which the values are all 10-fold lower (e.g., <3 mg mmol⁻¹ for stage A1).

Because the kidneys are a major source of the main growth factor for the proliferation of erythrocyte precursors, erythropoietin, advanced renal disease is often associated with an abnormally low number of erythrocytes and blood hemoglobin, manifestations of anemia. Because inorganic phosphate cannot be excreted, hyperphosphatemia is an important problem that has adverse consequences for bone health and often leads to an increase in parathyroid hormone known as secondary hyperparathyroidism. A diet low in phosphorus is considered mandatory for human patients undergoing dialysis, and stoichiometric binders of dietary phosphate are often prescribed to induce phosphate excretion in stool.

Inhibition of SGLT2 in diabetic humans has been found to produce modest elevations in BUN and serum creatinine, which early in the history of the development of SGLT2 inhibitors were interpreted as potentially adverse effects. However, the apparent decrease in eGFR has been found to be reversible and appears to represent an influence of SGLT2 inhibition on tubuloglomerular feedback resulting in decreased glomerular flow that is renoprotective over a sufficiently great period of time.

In a clinical trial evaluating the effects of the SGLT2 inhibitor canagliflozin in diabetic subjects with stage A3 albuminuria (all of whom were being treated with angiotensin system inhibitors), after a median period of 2.62 years, an interim assessment of safety and efficacy established that the trial should be halted because the continued administration of placebo was no longer justified (Perkovic et al., 2019, N Engl J Med 380:2295; doi: 10.1056/NEJMoa1811744). The risk of suffering any of the composite primary endpoint triad of end-stage kidney disease, a doubling of the serum creatinine level, or death from renal or cardiovascular causes, was 30% lower in the canagliflozin group than in the placebo group. Subjects assigned to the active arm experienced a prompt reduction in UACr that was evident at the first measurement at 6 months and remained approximately constant for up to 42 months. The eGFR of participants in the active arm dropped immediately, but then declined at a substantially slower rate than that for the participants in the placebo arm, and by study conclusion was higher for subjects who had been randomized to the active arm for the longest time than for the corresponding subjects in the control arm.

That these findings were neither specific to canagliflozin nor peculiar to the population chosen for study was supported by a meta-analysis of the effects of the SGLT2 inhibitors canagliflozin, dapagliflozin or empagliflozin that showed that SGLT2 inhibition decreased the likelihood of dialysis, transplantation, or death from renal disease by 33% compared to placebo, with good consistency observed between studies (Neuen et al., 2019, Lancet Diabetes Endocrinol 7:845; doi: 10.1016/S2213-8587(19)30256-6). Baseline albuminuria and use of angiotensin system inhibitors, enrollment criteria for the canagliflozin study (Perkovic et al., op. cit.), were not prerequisites for the renoprotective effect (Neuen et al., op. cit.).

### Canine CKD

Canine CKD has many similarities to human disease. Clinical signs that reflect decreased renal function may include evidence of systemic malaise and toxemia/azotemia in the form of one or more of anorexia, nausea, vomiting and weight loss. Signs of dehydration may also be present. Laboratory testing, in addition to identifying elevated creatinine, BUN and SDMA, may reveal acidosis, hypokalemia (low potassium) and anemia.

The management of canine CKD recommended by IRIS emphasizes control of hypertension in stage 1 disease by renin-angiotensin system (RAS) antagonists, followed by calcium channel blockers if RAS antagonists alone cannot produce an adequate hypotensive effect (http://www.iris-kidney.com/pdf/IRIS-DOG-Treatment_Recommendations_2019.pdf). In stage 2 more intensive intervention to control hypertension may be necessary and a renal diet should be begun if the urinary protein to creatinine ratio signifies borderline pathology (0.2 to 0.5) or frank proteinuria (ratio of UP/C>0.5). Anti-platelet agents (aspirin or clopidogrel) should be initiated if the serum albumin is <2.0 g dL⁻¹. Phosphate binders can be initiated if the serum phosphorus is ≥4.6 mg dL⁻¹. In stages 3 and 4, renal diets should be prescribed and the urine protein to creatinine ratio routinely measured. Metabolic acidosis should be managed with sodium bicarbonate or potassium citrate (if hypokalemia is present). Nausea and vomiting should be managed with anti-emetics. Omeprazole should be used to reduce gastric acidity if gastric bleeding or emesis-associated esophagitis is suspected. In stage 4 dialysis or renal transplantation can be considered. A feeding tube may be required to prevent malnutrition and/or dehydration

There is relatively little experimental support for the effectiveness of the angiotensin converting enzyme (ACE) inhibitor benazepril for canine CKD, however. In a blinded clinical trial enrolling 49 client-owned dogs with a primary endpoint measuring the difference in renal survival time between the placebo and benazepril cohorts, significance was not realized (King et al., 2017, J Vet Intern Med 31:1113; doi: 10.1111/jvim.14726). Renal survival time was the time to a composite of death, euthanasia or administration of parenteral fluids for renal failure. Although the primary endpoint was not met, proteinuria, as measured by the urinary protein to creatinine ratio, was significantly improved in the benazepril arm (p=0.0032).

### Feline CKD

Feline and canine CKD are managed pharmacologically by very similar strategies. A calcium channel blocker (CCB) or renin-angiotensin system antagonist is generally prescribed. A comparison of the angiotensin II receptor blocker (ARB) telmisartan with benazepril showed that telmisartan was noninferior to benazepril and produced a reduction in the urinary protein to creatinine ratio that was significant at all time points (7, 30, 60, 90, 120 and 180 days) whereas the change for benazepril was not significant (Sent et al. 2015 J Vet Intern Med 29:1479. doi: 10.1111/jvim.13639).

The management of feline CKD recommended by IRIS emphasizes control of hypertension in stage 1 disease by the CCB amlodipine or the ARB telmisartan (http://www.iris-kidney.com/pdf/IRIS_CAT_Treatment_Recommendations_2019.pdf). Use of renin-angiotensin-aldosterone system (RAAS) blockers is contraindicated in the setting of dehydration or hypovolemia. Cats that show urinary protein loss and low plasma protein levels may be at risk for thrombosis as in dogs, although there is relatively little published literature to support this. Cats in stage 2 should be managed as in stage 1, but if proteinuria is present, a renal diet should be initiated. If plasma phosphate levels remain intractably high, phosphate binders should be administered. In stage 3 a renal diet should always be prescribed, and phosphate binders may be needed. Metabolic acidosis should be managed with bicarbonate or potassium citrate as in dogs. Anti-emetics and appetite stimulants such as mirtazapine may be required. Intravenous fluids may be needed to control dehydration. Treatment of anemia with erythropoietin may be required. In stage 4 disease a feeding tube may be necessary, as well as routine administration of intravenous fluids. Dialysis or renal transplantation can be considered.

### Heart Failure

Heart failure is a syndrome with diverse etiologies resulting in a common manifestation. Heart failure can develop following many conditions or events, for example following chronic adrenergic stimulation, or as a result of genetic factors, as a consequence of infection by cardiotropic viruses, as a manifestation of advanced trypanosomal disease, as an outcome of irreversible ischemic damage, such as follows a myocardial infarction, as a result of deterioration of function of the mitral or aortic valve, as a result of autoimmune disease, such as an autoimmune myocarditis, or as a result of poorly understood spontaneous syndromes, such as hypertrophic cardiomyopathy.

The organismic consequences of a failing heart are for the most part similar. The inability to supply an appropriate pressure differential across the vasculature results in a fluctuating peripheral edema and a poor tissue perfusion that has consequences for both support of vital organs and resistance to infection. Limitation of the ability both to appropriately perfuse the lung and to distribute the oxygenated blood supply to other organs results in a chronic hypoxia that must be managed by provision of air enriched in oxygen, or even to 100% oxygen in severe cases. The peripheral edema increases the back pressure on the heart, taxing it further and leading to or exacerbating pre-existing hypertension. In many cases, the constant strain on the heart causes an adaptive hypertrophic response but the enlarged heart does not have a commensurately improved performance, and the overall vitality of the organism dwindles. Secondary adverse cardiovascular outcomes, such as myocardial infarct, atrial and/or ventricular fibrillation and stroke are common, and the risk of cardiovascular death is greatly elevated.

In humans the accumulation of peripheral interstitial fluid in heart failure is managed therapeutically with diuretics, particularly members of the loop diuretic class, which block the Na—K—Cl cotransporter 2 (NKCC2) expressed in the kidney in the thick ascending limb of the loop of Henle. NKCC2 cotransports one Na⁺, one K⁺ and 2 Cl⁻ ions in an electroneutral reabsorption of critical filtrate electrolytes. It is the most important transporter for the reabsorption of sodium ions. Management of exacerbations of heart failure in humans is typically carried out by intravenous infusion of loop diuretics. The most widely used loop diuretic in the US is furosemide, whereas torsemide is more frequently prescribed in the EU. Bumetanide is another commonly prescribed loop diuretic.

Other medications typically prescribed for heart failure in humans include RAS inhibitors, which predominantly consist of ACE inhibitors and ARBs, which respectively prevent the formation of angiotensin II and block its effect on angiotensin II receptor 1. These medications reduce blood pressure and are renoprotective, and have been shown to provide a survival benefit in heart failure.

Another common class of medication that is often co-prescribed in heart failure is a β-adrenergic receptor blocker, usually a β₁-selective agent such as atenolol or metoprolol or the mixed β and α receptor antagonist carvedilol. These medications protect against exacerbation of heart failure caused by chronic adrenergic stimulation. The latter can be caused by sympathetic nervous system adaptive responses to low cardiac output that are ultimately destructive as they increasingly tax the failing heart.

The body also mobilizes endogenous compensatory mechanisms in an attempt to effect sodium homeostasis. Typically, in advanced disease the plasma concentration of atrial natriuretic peptide is greatly increased. As its name suggests, atrial natriuretic peptide is a peptide hormone produced predominantly in the right atrium of the heart that increases the secretion of sodium by the kidneys. It is induced by stress in the atrial wall resulting from inadequate ventricular outflow and/or back pressure on the atrium and has effects at multiple locations in the kidneys by multiple mechanisms, including actions on NKCC2.

Because elimination of sodium ions is central to the homeostatic response to heart failure, drugs that produce natriuresis are especially important for the management of heart failure. Because reuptake of glucose by SGLT family transporters is accompanied by cotransport of sodium ions, SGLT inhibitors allow both glucose and sodium to remain in the tubular filtrate and thus affect sodium balance in the nephron.

Recently it has been demonstrated that the SGLT2 inhibitor dapagliflozin improves survival in human trial participants with heart failure and diabetes (Wiviott et al., 2019, N Engl J Med 380:347; doi: 10.1056/NEJMoa1812389) or with heart failure with and without diabetes (McMurray et al., 2019, N Engl J Med; doi: 10.1056/NEJMoa1911303). In the latter study a treatment effect of similar magnitude could be observed whether the subjects had diabetes or not. The protective effect of SGLT2 inhibition in heart failure appears to be a class effect, as it has been demonstrated in humans for canagliflozin and empagliflozin as well.

Heart failure in humans is often classified by the New York Heart Association scale, which has four levels of severity, I through IV, with level I representing health or minimal disease. This scale is often referenced in veterinary medicine, with the following interpretations: an animal with modified New York Heart Association (NYHA) Class II heart failure has fatigue, shortness of breath, coughing, etc., apparent when ordinary exercise is exceeded; an animal with modified NYHA Class III heart failure is comfortable at rest, but exercise capacity is minimal; and an animal with modified NYHA Class IV heart failure has no capacity for exercise and disabling clinical signs are present even at rest.

### Canine Heart Failure

Canine heart failure (Beaumier et al., 2018, J Vet Intern Med 32: 944; doi: 10.1111/jvim.15126) is most frequently caused by mitral valve disease, which is also common in humans. The mitral valve lies between the left atrium and left ventricle and prevents backflow from the ventricle into the atrium when the ventricle contracts. The most common form of mitral valve disease in dogs and humans is a chronic condition called myxomatous degeneration, in which the valve annulus becomes stretched and the chordae, the connective tissue structures holding the valve leaflets in place, become elongated. The leaflets themselves are often thickened, and loss of valvular integrity results in prolapse on systole (ventricular contraction) which, with increasing disease severity, results in mitral regurgitation, or backflow of blood into the left atrium. If the mitral valve is compromised to the point of regurgitation, the cardiac output per stroke decreases. The fraction of blood in the ventricle that enters the aorta upon systole is known as the ejection fraction. A reduced ejection fraction is a common characteristic of heart failure, although forms of heart failure in humans with preserved ejection fraction are frequently observed clinically.

Dogs in heart failure typically exhibit pulmonary edema and varying degrees of skeletal muscle loss that in its most severe manifestation is a form of cachexia, a life-threatening starvation-like syndrome characterized by extreme muscle wasting. In one series of 54 dogs in advanced heart failure, median survival was 281 days and at the time of the diagnosis of stage D disease (the most severe), 64% of the animals exhibited cachexia (Beaumier et al., op. cit.). Dogs receiving elevated doses of furosemide fared better than dogs receiving lower doses in this study.

Renal functional decline reflected by increased BUN is a common comorbidity of canine heart failure, and can limit the maximum dose of some medications that can be employed.

Canine heart failure is usually managed with furosemide combined with an ACE inhibitor (frequently enalapril or benazepril), pimobendan (a potentiator of myocardial contraction that also causes peripheral vasodilation), and sometimes spironolactone (a potassium-sparing diuretic that also protects against aldosterone-mediated cardiac fibrosis).

Pimobendan has been shown to extend the survival of dogs with stage C heart failure compared to benazepril in a single blind study (Häggström et al., 2008, J Vet Intern Med 22:1124; doi: 10.1111/j.1939-1676.2008.0150.x). The primary endpoint was a composite of either cardiac death, euthanasia for heart failure, or a treatment failure causing the investigator to remove the dog from the trial. The latter could be prompted by any of the following: persistent dyspnea, progressive ascites, severe cardiac cachexia, or severe exercise intolerance (attributable to a cardiac cause), despite receiving or failing to tolerate a diuretic dosage of furosemide (12 mg kg⁻¹ day⁻¹) and spironolactone (6 mg kg⁻¹ day⁻¹). The median survival was 267 days for dogs assigned to the pimobendan group and 140 days for dogs assigned to the benazepril group, with a hazard ratio of 0.688, 95% CI 0.516 to 0.916, p=0.0099.

Pimobendan has also been shown to extend the survival of dogs with stage B heart failure compared to placebo in a much longer study (Boswood et al., 2016, J Vet Intern Med 30:176; doi: 10.1111/jvim.14586) that had a composite primary endpoint of the onset of congestive heart failure (CHF), cardiac-related death, or euthanasia. In the pimobendan cohort the median time to the primary endpoint was 1228 days (95% CI: 856-NA), whereas the median time to primary endpoint was 766 days (95% CI: 667-875) in the placebo group (p=0.0038). The hazard ratio for the pimobendan group was 0.64 (95% CI: 0.47-0.87) compared with the placebo group.

### Feline Heart Failure

Feline heart failure is usually the result of hypertrophic cardiomyopathy, a condition in cats that is very similar to the analogous condition in humans, but that has a much higher prevalence in cats, affecting 10-15% of the population (Freeman et al., 2017, Cardiol Res 8:139; doi: 10.14740/cr578w). Feline hypertrophic cardiomyopathy is often asymptomatic, but can have severe consequences. The signs of hypertrophic cardiomyopathy are sudden death, syncope (abrupt loss of consciousness), congestive heart failure and aortic thromboembolism (clot formation in the aorta). After diagnosis, most cats die of heart failure, embolism or sudden death. As in dogs, cachexia is a common comorbidity of heart failure in cats.

Management of symptomatic feline hypertrophic cardiomyopathy is similar to that of canine heart failure, with furosemide and RAS inhibitors (usually enalapril or benazepril) frequently recommended. However, a recent report of a prospective, randomized blinded study of the ACE inhibitor benazepril in 151 client-owned cats with heart disease confirmed by echocardiography showed no benefit for the primary endpoint of treatment failure (King et al., 2019, J Vet Intern Med; doi: 10.1111/jvim.15572). Anti-platelet agents such as aspirin or clopidogrel are often prescribed to reduce the risk of aortic thromboembolism. Pimobendan, although not approved for use in cats, is often mentioned in veterinary publications on feline heart failure, together with digoxin, another positive inotrope (agent that increases the strength of heart contractions). As in the management of canine disease, spironolactone is also a frequently prescribed medication.

### Hypertension

Hypertension is a widespread but poorly understood disease. The vast majority of human hypertension has no known cause and is referred to as primary or essential hypertension. Essential hypertension is often observed in the setting of atherosclerotic cardiovascular disease, which is associated with changes to the arteries that result in stiffening and decreased compliance that are widely believed to contribute to the severity and rate of progression of the disease but a direct demonstration that atherosclerosis causes hypertension has not been made. Inadequately treated hypertension has one of the largest estimated adverse effects on human longevity because of the prevalence of the condition.

The current understanding of canine and feline hypertension is incomplete. As in humans the canine or feline disease is observed in the setting of other conditions, such as CKD or heart failure, but establishing causation can be challenging. The kidney, for example, is both a target of the adverse effects of hypertension on organ systems as well as a critical organ for the maintenance of blood pressure. Dysregulation of fluid balance in heart failure increases peripheral load and taxes the failing heart, which must work harder, exacerbating the hypertension.

### II. Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

As used herein, the terms “a,” “an,” or “the”, not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

As used herein, the terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

Any ranges used herein, for example “from 5 to 100” are meant to include both endpoints of the stated range, as well as all intermediate ranges even though not specifically stated. The range “from 5 to 100” also includes, for example “5 to 90”, “10 to 100”, “22 to 32” and the like.

The inferred pharmacokinetic parameters of a noncompartmental analysis are defined as most frequently employed in the art.

“AUC” is the “area under the curve” of the plasma concentration as a function of time, constructed by the linear trapezoidal rule, according to which the AUC is given by the summation of the arithmetic mean of the concentration at two adjacent sampling points in time, multiplied by the difference in time between those sampling points: (C(tᵢ)+C(tᵢ₊₁))(tᵢ₊₁−tᵢ)/2.

“AUC₀₋ₜ” represents the AUC from time 0 to the last quantifiable concentration.

“AUC_{0-∞}” represents the AUC from time 0 to infinity, as produced by extrapolation of a simple (monophasic) exponential decay. AUC_{0-∞}=AUC₀₋ₜ+Cₗₐₛₜ/kₑₗ, where Cₗₐₛₜ is the last quantifiable concentration and kₑₗ is the terminal elimination rate constant.

“Cₘₐₓ” is the greatest observed plasma concentration.

“Tₘₐₓ” is the time at which the greatest observed plasma concentration is recorded and, when presented for a population, is, unless otherwise described, given as the population median.

All other pharmacokinetic parameters, when presented for a population, are given as either the arithmetic or the geometric mean.

“t_{1/2}” is the terminal half-life, also referred to as the elimination half-life. If the empirically determined terminal elimination kinetics are not first-order in time, t_{1/2} cannot be defined. t_{1/2}=−ln(2)/kₑₗ≈0.693/kₑₗ.

In addition, the pharmacodynamic quantities associated with a simple logistic (Hill coefficient=1) are defined as typically encountered in the art.

“ED₅₀” is the estimated dose at which 50% of the maximum projected effect is reached.

“Eₘₐₓ” is the projected maximal effect.

“CI” is the abbreviation for confidence interval, a 95% interval unless noted otherwise.

“SD” is the abbreviation for standard deviation.

“SEM” is the abbreviation for standard error of measurement.

As used herein, unless otherwise indicated, the term “alkyl” alone or in combination refers to a monovalent saturated aliphatic hydrocarbon radical having the indicated number of carbon atoms. The radical may be a linear or branched chain. Illustrative examples of alkyl groups include, but are not limited to, methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, n-dodecyl, n-tetradecyl, n-hexadecyl, n-octadecyl, n-eicosyl and the like. Preferred alkyl groups include methyl, ethyl, n-propyl and isopropyl.

As used herein, unless otherwise indicated, the term “alkenyl” alone or in combination refers to a monovalent aliphatic hydrocarbon radical having the indicated number of carbon atoms and at least one carbon-carbon double bond. The radical may be a linear or branched chain, in the E or Z form. Illustrative examples of alkenyl groups include, but are not limited to, vinyl, 1-propenyl, 2-propenyl, isopropenyl, 1-butenyl, 2-butenyl, isobutenyl, 2-methyl-1-propenyl, 1-pentenyl, 2-pentenyl, 4-methyl-2-pentenyl, 1,3-pentadienyl, 2,4-pentadienyl, 1,3-butadienyl and the like. Preferred alkenyl groups include vinyl, 1-propenyl and 2-propenyl.

As used herein, unless otherwise indicated, the term “alkynyl” alone or in combination refers to a monovalent aliphatic hydrocarbon radical having the indicated number of carbon atoms and at least one carbon-carbon triple bond. The radical may be a linear or branched chain. Illustrative examples of alkynyl groups include, but are not limited to, ethynyl, 1-propynyl, 2-propynyl, 1-butynyl, 2-butynyl, 1-pentynyl, 2-pentynyl, 3-methyl-1-pentynyl, 3-pentynyl, 1-hexynyl, 2-hexynyl, 3-hexynyl and the like. Preferred alkynyl groups include ethynyl, 1-propynyl and 2-propynyl.

As used herein, unless otherwise indicated, the term “cycloalkyl” alone or in combination refers to a monovalent alicyclic saturated hydrocarbon radical having three or more carbons forming a carbocyclic ring. Illustrative examples of cycloalkyl groups include, but are not limited to, cyclopropyl, cyclobutyl, cyclopentyl, cyclohexyl, cycloheptyl, cyclooctyl, cyclononyl and the like.

As used herein, unless otherwise indicated, the term “cycloalkenyl” alone or in combination refers to a monovalent alicyclic hydrocarbon radical having three or more carbons forming a carbocyclic ring and at least one carbon-carbon double bond. Illustrative examples of cycloalkenyl groups include, but are not limited to, cyclopentenyl, cyclohexenyl and the like.

As used herein, unless otherwise indicated, the term “alkylene” refers to a divalent hydrocarbon radical that is formed by removal of a hydrogen atom from an alkyl radical, as such term is defined above.

As used herein, unless otherwise indicated, the term “aryl” alone or in combination refers to a monovalent aromatic hydrocarbon radical having six to ten carbon atoms forming a carbocyclic ring. Illustrative examples of aryl groups include, but are not limited to, phenyl, naphthyl, tetrahydronaphthyl, indanyl and the like. Preferred aryl groups are phenyl and naphthyl, optionally mono- or disubstituted by identical or different substituents selected from halo, cyano, C₁-C₃ alkyl, C₃-C₆ cycloalkyl, difluoromethyl, trifluoromethyl, C₁-C₃ alkoxy, difluoromethoxy and trifluoromethoxy.

As used herein, the term “halo” means a monovalent halogen radical or atom selected from fluoro, chloro, bromo and iodo. Preferred halo groups are fluoro, chloro and bromo.

As used herein, unless otherwise indicated, the term “heterocycloalkyl” alone or in combination refers to a cycloalkyl group as defined above in which one or more carbons in the ring is replaced by a heteroatom selected from N, S and O. Accordingly, a C₃-C₆ heterocycloalkyl group is a three- to six-membered ring in which one or more of the carbon atom ring vertices has been replaced by N, S or O. Illustrative examples of heterocycloalkyl groups include, but are not limited to, pyrrolidinyl, tetrahydrofuranyl, piperazinyl, tetrahydropyranyl, and the like.

As used herein, unless otherwise indicated, the term “heteroaryl” alone or in combination refers to a monovalent aromatic heterocyclic radical having two to nine carbons and one to four heteroatoms selected from N, S and O forming a five- to ten-membered monocyclic or fused bicyclic ring. Illustrative examples of heteroaryl groups include, but are not limited to, pyridyl, pyridazinyl, pyrazinyl, pyrimidinyl, triazinyl, quinolinyl, isoquinolinyl, quinoxalinyl, quinazolinyl, benzotriazinyl, benzimidazolyl, benzopyrazolyl, benzotriazolyl, benzisoxazolyl, isobenzofuryl, isoindolyl, indolizinyl, thienopyridinyl, thienopyrimidinyl, pyrazolopyrimidinyl, imidazopyridines, benzothiaxolyl, benzofuranyl, benzothienyl, indolyl, isothiazolyl, pyrazolyl, indazolyl, imidazolyl, triazolyl, tetrazolyl, oxazolyl, isoxazolyl, oxadiazolyl, thiadiazolyl, pyrrolyl, thiazolyl, furyl, thienyl and the like. Five- or six-membered monocyclic heteroaryl rings include: tetrahydrothiophenyl, pyridyl, pyridazinyl, pyrazinyl, pyrimidinyl, triazinyl, isothiazolyl, pyrazolyl, imidazolyl, triazolyl, tetrazolyl, oxazolyl, isoxazolyl, oxadiazolyl, thiadiazolyl, pyrrolyl, thiazolyl, furyl, thienyl and the like. Eight- to ten-membered bicyclic heteroaryl rings having one to four heteroatoms include: quinolinyl, isoquinolinyl, quinoxalinyl, quinazolinyl, benzotriazinyl, benzimidazolyl, benzopyrazolyl, benzotriazolyl, benzisoxazolyl, isobenzofuryl, isoindolyl, indolizinyl, thienopyridinyl, thienopyrimidinyl, pyrazolopyrimidinyl, imidazopyridinyl, benzothiaxolyl, benzofuranyl, benzothienyl, indolyl, indazolyl, and the like.

As used herein, unless otherwise indicated, the term “alkoxy” alone or in combination refer to an aliphatic radical of the form alkyl-O—, wherein alkyl is as defined above. Illustrative examples of alkoxy groups include, but are not limited to, methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy, octoxy and the like. Preferred alkoxy groups include methoxy and ethoxy.

As used herein, unless otherwise indicated, the term “cycloalkoxy” alone or in combination refer to an aliphatic radical of the form cycloalkyl-O—, wherein cycloalkyl is as defined above. Illustrative examples of cycloalkoxy groups include, but are not limited to, cyclopropoxy, cyclobutoxy and cyclopentoxy.

As used herein, unless otherwise indicated, the term “heterocycloalkoxy” alone or in combination refer to an aliphatic radical of the form heterocycloalkyl-O—, wherein heterocycloalkyl is as defined above. Illustrative examples of heterocycloalkoxy groups include, but are not limited to, tetrahydrofuranoxy, pyrrolidinoxy and tetrahydrothiophenoxy.

As used herein, unless otherwise indicated, the term “haloalkyl” refers to an alkyl radical as described above substituted with one or more halogens. Illustrative examples of haloalkyl groups include, but are not limited to, chloromethyl, dichloromethyl, fluoromethyl, difluoromethyl, trifluoromethyl, 2,2,2-trichloroethyl and the like.

As used herein, when a particular position in a compound is designated as being “deuterated” (the element deuterium is represented by the letter “D” in chemical structures and formulas and indicated with a lower case “d” in chemical names, according to the Boughton system), it is understood that the abundance of deuterium at that position is substantially greater than the natural abundance of deuterium, which is 0.015%. In certain embodiments, a composition has a minimum isotopic enrichment factor of at least 5 (0.075% deuterium incorporation), e.g., at least 10 (0.15% deuterium incorporation). In other embodiments, a composition has an isotopic enrichment factor of at least 50 (0.75% deuterium incorporation), at least 500 (7.5% deuterium incorporation), at least 2000 (30% deuterium incorporation), at least 3000 (45% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), or at least 6600 (99% deuterium incorporation).

As used herein, the term “prodrug” refers to a precursor compound that, following administration, releases the biologically active compound in vivo via some chemical or physiological process (e.g., a prodrug on reaching physiological pH or through enzyme action is converted to the biologically active compound). A prodrug itself may either lack or possess the desired biological activity.

As used herein, “treatment” or “treating,” “management” or “managing,” or “palliating” or “ameliorating” are used interchangeably herein. These terms refer to an approach for obtaining beneficial or desired results including but not limited to a therapeutic benefit. By therapeutic benefit is meant amelioration of the underlying disorder being treated. Also, a therapeutic benefit is achieved with the amelioration of one or more of the physiological symptoms associated with the underlying disorder such that an improvement is observed in the patient, notwithstanding that the patient may still be afflicted with the underlying disorder. Treatment includes causing the clinical symptoms of the disease to slow in development by administration of a composition; suppressing the disease, that is, causing a reduction in the clinical symptoms of the disease; inhibiting the disease, that is, arresting the development of clinical symptoms by administration of a composition after the initial appearance of symptoms; and/or relieving the disease, that is, causing the regression of clinical symptoms by administration of a composition after their initial appearance.

As used herein, the term “effective amount” or “therapeutically effective amount” includes an amount or quantity effective, at dosages and for periods of time necessary, to produce a desired (e.g., therapeutic or prophylactic) result with respect to the indicated disease, disorder, or condition. The desired result may comprise a subjective or objective improvement in the recipient of the effective amount. The effective amount will vary with the type of companion being treated. Efficacy can also be expressed as “-fold” increase or decrease. For example, a therapeutically effective amount can have at least a 1.2-fold, 1.5-fold, 2-fold, 5-fold, or more effect over a control.

As used herein, “preselected” refers to the selection of one or more clinical, behavioral, and/or physiological criteria in a subject prior to initiating a therapeutic treatment. One or more clinical criteria can include the selection of conditions or diseases in a companion animal. One or more clinical criteria can also include the absence of one or more conditions or diseases in a companion animal. For example, a companion animal who is preselected to not have a particular disease was not diagnosed or was not exhibiting symptoms of said disease prior to initiating a therapeutic treatment. As an additional example, a companion animal who is preselected based on one or more behavioral criteria was practicing or exhibiting said behavioral criteria prior to initiating a therapeutic treatment.

As used herein, the term “heart failure” refers to a condition that can result from any structural or functional cardiac disorder that impairs the ability of the heart to fill with or pump a sufficient amount of blood throughout the body. Heart failure can develop following many conditions or events, for example following chronic adrenergic stimulation, or as a result of genetic factors, as a consequence of infection by cardiotropic viruses, as a manifestation of advanced trypanosomal disease, as an outcome of irreversible ischemic damage, such as follows a myocardial infarction, as a result of deterioration of function of the mitral or aortic valve, as a result of autoimmune disease, such as an autoimmune myocarditis, or as a result of poorly understood spontaneous syndromes, such as hypertrophic cardiomyopathy. Recognized forms of heart failure include, but are not limited to valvular heart disease (such as mitral valve disease, aortic valve disease, or atrioventricular valvular insufficiency), ischemic heart diseases, congenital heart diseases, dilated cardiomyopathy, hypertrophic cardiomyopathy, atrial septal defect, ventricular septal defect and symptomatic heart disease. Specifically, the valvular heart disease of the present disclosure includes valvular insufficiencies such as aortic regurgitation, aortic stenosis, mitral regurgitation and mitral stenosis.

As used herein, the term “chronic kidney disease” (CKD) refers to an indolent progressive condition characterized by progressive loss in renal function over a period of months or years. Generally, CKD affects the kidney through destruction of the renal parenchyma and the loss of functional nephrons or glomeruli. CKD can result from different causes, but the final pathway remains renal fibrosis. Exemplary etiology of CKD includes, but is not limited to, cardiovascular diseases, hypertension, diabetes, glomerulonephritis, polycystic kidney diseases, and kidney graft rejection.

As used herein, “blood pressure” refers to pressure exerted on the walls of blood vessels by blood that is pumped out of the heart and flows in the blood vessels. Generally, a subject's blood pressure is recorded as the systolic pressure (heart contraction) in mm Hg followed by the diastolic pressure (heart relaxation) in mm Hg (e.g. 120/88 mm Hg). The quantified pressures provided are usually the mean pressure over the course of multiple heart beats (i.e., more than one).

As used herein “pulse pressure” refers to the difference between the systolic and diastolic blood pressures.

As used herein “durable response” includes adequate relief of symptoms throughout the treatment regimen, and continuous adequate relief of symptoms throughout the treatment regimen. The duration of the durable response can be, for example, 2 weeks, 3 weeks, 4 weeks, 5 weeks, 6 weeks, 7 weeks, 8 weeks, 24 weeks, 48 weeks, 96 weeks or as long as the subject continues the treatment regimen.

### III. Detailed Description of Embodiments

As mentioned above, the current application is drawn to the discovery that despite their initial development for the use in treating metabolic disorders such as type 2 diabetes, sodium-glucose linked transporter (SGLT) inhibitors can be used in the management of hypertension, renal disease (e.g., chronic kidney disease) or heart failure in companion animals (in particular, felines and canines).

A. Sodium-Glucose Linked Transporter (SGLT) Inhibitors

A variety of compounds that inhibit a sodium-dependent glucose transporter (SGLT) as well as prodrugs thereof are known in the art, each of which can be used in the presently described methods.

SGLT inhibitors have been or are being developed to treat human metabolic disorders including type 2 diabetes and allied conditions. Among them are atigliflozin, bexagliflozin, canagliflozin, dapagliflozin, empagliflozin, enavogliflozin, ertugliflozin, henagliflozin, ipragliflozin, janagliflozin, licogliflozin, luseogliflozin, mizagliflozin, remogliflozin, sergliflozin, sotagliflozin, tianagliflozin and tofogliflozin. In addition, velagliflozin is being developed for the management of animal forms of diabetes and related morbidities.

SGLT inhibitors produce glucosuria but not hypoglycemia and are expected to produce changes in sodium ion balance in the renal proximal tubule that may have consequences downstream of their location in the nephron. Because the SGLT1 transporter has a 2:1 sodium ion to glucose stoichiometry for reuptake, dual inhibition of both SGLT1 and SGLT2 is expected to have greater effects on natriuresis than inhibition of SGLT2 alone. Inhibition of SGLT1 alone is not expected to produce substantial changes to tubular electrolyte homeostasis because humans lacking SGLT1 usually do not exhibit glucosuria, indicating that SGLT2 can nearly completely compensate for a loss of SGLT1.

Many of the SGLT inhibitors are selective for human SGLT2 compared to human SGLT1. Others have lower selectivity, such as licogliflozin or sotagliflozin, and some are more active against SGLT1 than SGLT2, such as mizagliflozin. Selectivity can be species-dependent, so that high selectivity for human SGLT2 does not necessarily indicate high selectivity for, for example, cat or dog SGLT2. And depending on the disease indication a greater or lesser degree of inhibition of one or the other SGLT protein may be desirable.

In some embodiments, a compound that inhibits a sodium-dependent glucose transporter (SGLT) has a Formulae I, II, III, IV or V as defined below.

Accordingly, in one embodiment, the compound that inhibits SGLT for use in the present disclosure are compounds of Formula I:

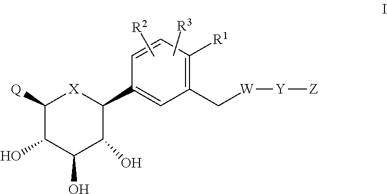

wherein

X represents oxygen or sulfur;

Q represents —CH₃, —CH₂OH, C₁-C₆ alkylsulfanyl, C₁-C₆ alkylsulfinyl, C₁-C₆ alkylsulfonyl, C₁-C₆ haloalkylsulfanyl, C₁-C₆ haloalkylsulfinyl, C₁-C₆ haloalkylsulfonyl, or —CH₂OV, where V represents (C₁-C₃ alkyl)oxycarbonyl, (C₁-C₆ alkyl)carbonyl, phenyloxycarbonyl, benzylcarbonyl or benzyloxycarbonyl;

R¹ represent hydrogen, halo, C₁-C₃ alkyl, C₂-C₃ alkynyl, C₃-C₆ cycloalkyl, hydroxy or cyano;

R² and R³ each independently represent hydrogen, halo, C₁-C₃ alkyl, C₂-C₃ alkynyl, C₃-C₆ cycloalkyl, hydroxy or cyano; or when R² and R³ are on adjacent ring vertices they can combine to form a 5-membered heterocycloalkyl comprising one to 2 heteroatoms as ring vertices selected from N, O, and S;

W represents a 5- to 6-membered aryl or heteroaryl ring, or an 8- to 10-membered fused bicyclic aryl or heteroaryl ring,

wherein W optionally may be mono- or disubstituted by identical or different substituents selected from halo, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl, and C₁-C₆ alkylsulfonyl; or when two substituents are on adjacent ring verticies they can combine to form a 6-membered heterocycloalkyl comprising one to two heteroatoms selected from N, O, and S, and
wherein alkyl groups or portions optionally may be partly or completely fluorinated;

Y represents a single bond or a 5- to 6-membered aryl or heteroaryl ring,

wherein Y optionally may be mono- or disubstituted by identical or different substituents selected from halo, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl, and C₁-C₆ alkylsulfonyl, and
wherein alkyl groups or portions optionally may be partly or completely fluorinated;

Z represents hydrogen, halo, hydroxy, cyano, C₁-C₃ alkyl, C₁-C₃ alkoxy, C₂-C₃ alkynyl, C₃-C₆ cycloalkyl, C₃-C₆ heterocycloalkyl, C₃-C₆ cycloalkoxy, C₃-C₆ heterocycloalkoxy, (C₁-C₃ alkoxy)C₁-C₃ alkoxy or (C₃-C₆ cycloalkoxy)C₁-C₃ alkoxy,

wherein alkyl, alkynyl, cycloalkyl and heterocycloalkyl groups or portions optionally may be partly or completely fluorinated and may be mono- or disubstituted by identical or different substituents selected from chloro, hydroxy, C₁-C₃ alkyl, C₁-C₃ alkoxy, cyano, —NR^{a}R^{b}, —C(O)NR^{a}R^{b}, C₁-C₆ alkylsulfanyl, C₁-C₆ alkylsulfinyl, and C₁-C₆ alkylsulfonyl;
wherein cycloalkyl groups are optionally substituted with from 1 to 2 substituents selected from hydrogen, halo, hydroxy, cyano, C₁-C₃ alkyl, C₁-C₃ alkoxy; or when the two substituents are on adjacent ring vertices they can combine to form a 3- to 5-membered cycloalkyl;

R^{a} and R^{b} each independently represent hydrogen or C₁-C₆ alkyl, wherein alkyl groups optionally may be partly or completely fluorinated; and

wherein optionally one or more hydrogen atoms may be substituted with deuterium.

In certain preferred embodiments of compounds of Formula I for use in the disclosure, X represents oxygen or sulfur; Q represents —CH₂OH or methylsulfonyl; R¹ represents hydrogen, chloro, fluoro, methyl or cyano; R² represents hydrogen or hydroxy; W represents phenyl; Y represents a single bond; and Z represents ethyl, ethoxy, ethynyl, cyclopropyl, benzo[b]thiophen-2-yl, azulenyl, tetrahydrofuran-3-yloxy or cyclopropoxyethoxy.

In particularly preferred embodiments, compounds of Formula I for use in the present disclosure are selected from:

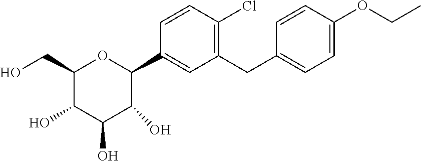 [SMILES: CCOc1ccc(Cc2cc([C@@H]3O[C@H](CO)[C@@H](O)[C@H](O)[C@H]3O)ccc2Cl)cc1]

### (2S,3R,4R,5S,6R)-2-(4-chloro-3-(4-ethoxybenzyl)phenyl)-6-(hydroxymethyl)tetrahydro-2H-pyran-3,4,5-triol

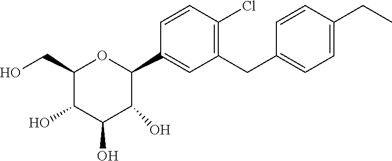 [SMILES: CCc1ccc(Cc2cc([C@@H]3O[C@H](CO)[C@@H](O)[C@H](O)[C@H]3O)ccc2Cl)cc1]

### (2S,3R,4R,5S,6R)-2-(4-chloro-3-(4-ethylbenzyl)phenyl)-6-(hydroxymethyl)tetrahydro-2H-pyran-3,4,5-triol

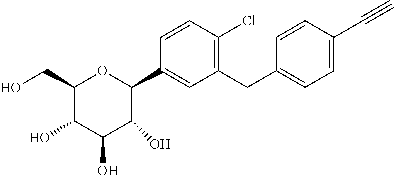 [SMILES: C#Cc1ccc(Cc2cc([C@@H]3O[C@H](CO)[C@@H](O)[C@H](O)[C@H]3O)ccc2Cl)cc1]

### (2S,3R,4R,5S,6R)-2-(4-chloro-3-(4-ethynylbenzyl)phenyl)-6-(hydroxymethyl)tetrahydro-2H-pyran-3,4,5-triol

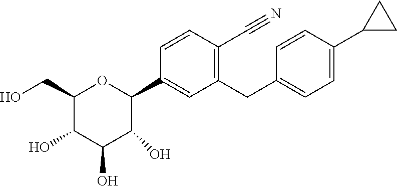 [SMILES: N#Cc1ccc([C@@H]2O[C@H](CO)[C@@H](O)[C@H](O)[C@H]2O)cc1Cc1ccc(C2CC2)cc1]

### 2-(4-cyclopropylbenzyl)-4-((2 S,3R,4R,5S,6R)-3,4,5-trihydroxy-6-(hydroxymethyl)tetrahydro-2H-pyran-2-yl)benzonitrile

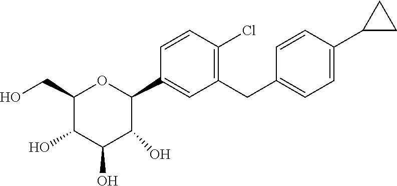 [SMILES: OC[C@H]1O[C@@H](c2ccc(Cl)c(Cc3ccc(C4CC4)cc3)c2)[C@H](O)[C@@H](O)[C@@H]1O]

### (2 S,3R,4R,5S,6R)-2-(4-chloro-3-(4-cyclo propylbenzyl)phenyl)-6-(hydroxymethyl)tetrahydro-2H-pyran-3,4,5-triol

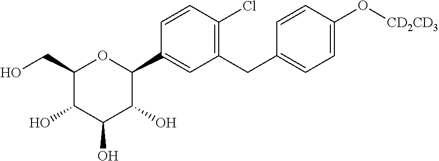

### (2S,3R,4R,5S,6R)-2-(4-chloro-3-(4-(ethoxy-d₅)benzyl)phenyl)-6-(hydroxymethyl)tetrahydro-2H-pyran-3,4,5-triol

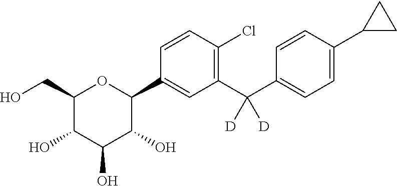 [SMILES: [2H]C([2H])(c1ccc(C2CC2)cc1)c1cc([C@@H]2O[C@H](CO)[C@@H](O)[C@H](O)[C@H]2O)ccc1Cl]

### (2S,3R,4R,5S,6R)-2-(4-chloro-3-((4-cyclopropylphenyl)methyl-d₂)phenyl)-6-(hydroxymethyl)tetrahydro-2H-pyran-3,4,5-triol

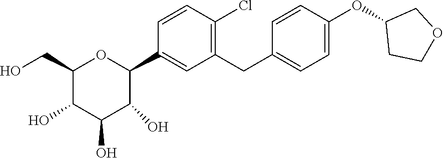 [SMILES: OC[C@H]1O[C@@H](c2ccc(Cl)c(Cc3ccc(O[C@H]4CCOC4)cc3)c2)[C@H](O)[C@@H](O)[C@@H]1O]

### (2S,3R,4R,5S,6R)-2-(4-chloro-3-(4-((S)-tetrahydrofuran-3-yloxy)benzyl)phenyl)-6-(hydroxymethyl)tetrahydro-2H-pyran-3,4,5-triol

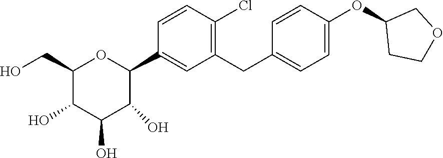 [SMILES: OC[C@H]1O[C@@H](c2ccc(Cl)c(Cc3ccc(O[C@@H]4CCOC4)cc3)c2)[C@H](O)[C@@H](O)[C@@H]1O]

### (2S,3R,4R,5S,6R)-2-(4-chloro-3-(4-((R)-tetrahydrofuran-3-yloxy)benzyl)phenyl)-6-(hydroxymethyl)tetrahydro-2H-pyran-3,4,5-triol

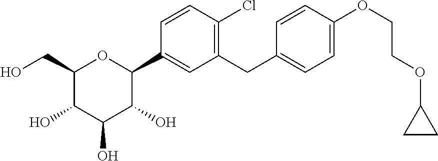 [SMILES: OC[C@H]1O[C@@H](c2ccc(Cl)c(Cc3ccc(OCCOC4CC4)cc3)c2)[C@H](O)[C@@H](O)[C@@H]1O]

### (2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol

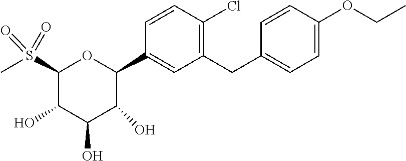 [SMILES: CCOc1ccc(Cc2cc([C@@H]3O[C@H](S(C)(=O)=O)[C@@H](O)[C@H](O)[C@H]3O)ccc2Cl)cc1]

### (2S,3R,4R,5 S,6R)-2-(4-chloro-3-(4-ethoxybenzyl)phenyl)-6-(methylsulfonyl)tetrahydro-2H-pyran-3,4,5-triol

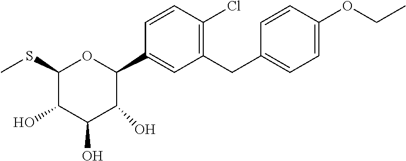 [SMILES: CCOc1ccc(Cc2cc([C@@H]3O[C@H](SC)[C@@H](O)[C@H](O)[C@H]3O)ccc2Cl)cc1]

### (2S,3R,4R,5S,6R)-2-(4-chloro-3-(4-ethoxybenzyl)phenyl)-6-(methylthio)tetrahydro-2H-pyran-3,4,5-triol

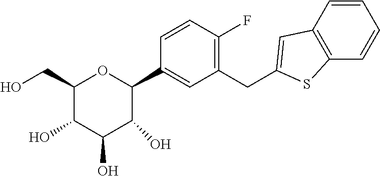 [SMILES: OC[C@H]1O[C@@H](c2ccc(F)c(Cc3cc4ccccc4s3)c2)[C@H](O)[C@@H](O)[C@@H]1O]

### (2S,3R,4R,5S,6R)-2-(3-(benzo[b]thiophen-2-ylmethyl)-4-fluorophenyl)-6-(hydroxymethyl)tetrahydro-2H-pyran-3,4,5-triol

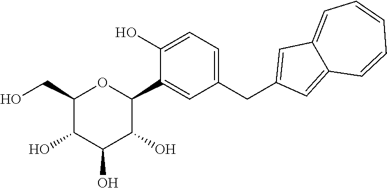 [SMILES: OC[C@H]1O[C@@H](c2cc(Cc3cc4cccccc-4c3)ccc2O)[C@H](O)[C@@H](O)[C@@H]1O]

### (2S,3R,4R,5S,6R)-2-(5-(azulen-2-ylmethyl)-2-hydroxyphenyl)-6-(hydroxymethyl)tetrahydro-2H-pyran-3,4,5-triol

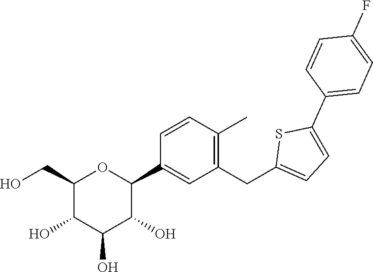 [SMILES: Cc1ccc([C@@H]2O[C@H](CO)[C@@H](O)[C@H](O)[C@H]2O)cc1Cc1ccc(-c2ccc(F)cc2)s1]

### (2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol

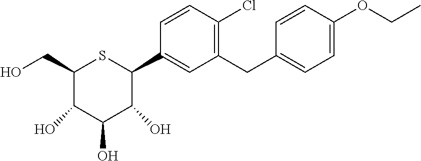 [SMILES: CCOc1ccc(Cc2cc([C@@H]3S[C@H](CO)[C@@H](O)[C@H](O)[C@H]3O)ccc2Cl)cc1]

### (2S,3R,4R,5S,6R)-2-(4-chloro-3-(4-ethoxybenzyl)phenyl)-6-(hydroxymethyl)tetrahydro-2H-thiopyran-3,4,5-triol

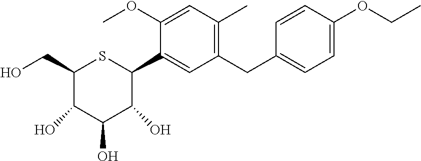 [SMILES: CCOc1ccc(Cc2cc([C@@H]3S[C@H](CO)[C@@H](O)[C@H](O)[C@H]3O)c(OC)cc2C)cc1]

### (2S,3R,4R,5S,6R)-2-(5-(4-ethoxybenzyl)-2-methoxy-4-methylphenyl)-6-(hydroxymethyl)tetrahydro-2H-thiopyran-3,4,5-triol

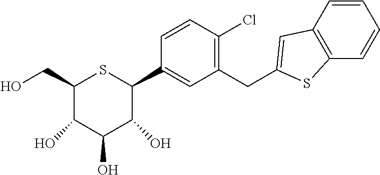 [SMILES: OC[C@H]1S[C@@H](c2ccc(Cl)c(Cc3cc4ccccc4s3)c2)[C@H](O)[C@@H](O)[C@@H]1O]

### (2S,3R,4R,5S,6R)-2-(3-(benzo[b]thiophen-2-ylmethyl)-4-chlorophenyl)-6-(hydroxymethyl)tetrahydro-2H-thiopyran-3,4,5-triol

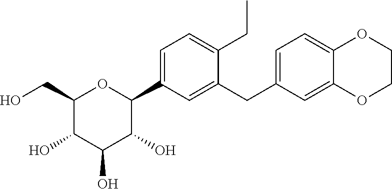 [SMILES: CCc1ccc([C@@H]2O[C@H](CO)[C@@H](O)[C@H](O)[C@H]2O)cc1Cc1ccc2c(c1)OCCO2]

### (2S,3R,4R,5S,6R)-2-[3-(2,3-Dihydro-benzo[1,4]dioxin-6-ylmethyl)-4-ethyl-phenyl]-6-hydroxymethyl-tetrahydro-pyran-3,4,5-triol

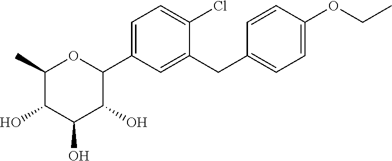 [SMILES: CCOc1ccc(Cc2cc(C3O[C@H](C)[C@@H](O)[C@H](O)[C@H]3O)ccc2Cl)cc1]

### (3R,4S,5S,6R)-2-(4-chloro-3-(4-ethoxybenzyl)phenyl)-6-methyltetrahydro-2H-pyran-3,4,5-triol

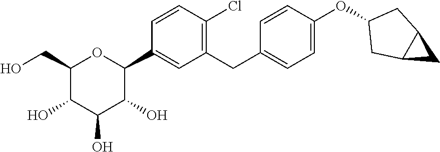 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

### (2S,3R,4R,5S,6R)-2-(3-(4-(((1R,3s,5S)-bicyclo[3.1.0]hexan-3-yl)oxy)benzyl)-4-chlorophenyl)-6-(hydroxymethyl)tetrahydro-2H-pyran-3,4,5-triol and

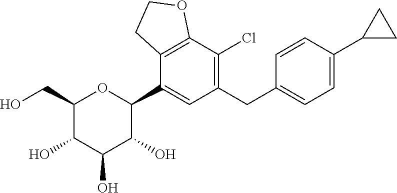 [SMILES: OC[C@H]1O[C@@H](c2cc(Cc3ccc(C4CC4)cc3)c(Cl)c3c2CCO3)[C@H](O)[C@@H](O)[C@@H]1O]

### (2S,3R,4R,5S,6R)-2-(7-chloro-6-(4-cyclopropylbenzyl)-2,3-dihydrobenzofuran-4-yl)-6-(hydroxymethyl)tetrahydro-2H-pyran-3,4,5-triol

In another aspect, the compound that inhibits SGLT for use in the present disclosure are compounds of Formula II:

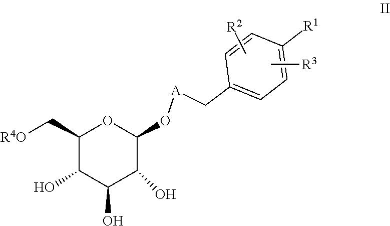

wherein

A represents a 5- to 6-membered aryl or heteroaryl ring,

wherein A optionally may be mono- or disubstituted by identical or different substituents selected from halo, hydroxy and C₁-C₆ alkyl, and
wherein alkyl groups or portions optionally may be partly or completely fluorinated;

R¹ represents C₁-C₃ alkoxy, wherein the alkyl portion optionally may be partly or completely fluorinated or substituted with —NH—C₁-C₄ alkyl-C(O)NH₂;

R² and R³ each independently represent hydrogen, halo or C₁-C₃ alkyl, wherein the alkyl group optionally may be partly or completely fluorinated; and

R⁴ represents hydrogen, (C₁_₆ alkyl)carbonyl, (C₁_₃ alkyl)oxycarbonyl, phenyloxycarbonyl, benzyloxycarbonyl or benzylcarbonyl.

In certain preferred embodiments of compounds of Formula II for use in the disclosure, A represents benzene, tetrahydrothiophene or 1-isopropyl-5-methyl-1H-pyrazole; R¹ represents methoxy, trifluoromethoxy or isopropoxy; R² and R³ each represent hydrogen; and R⁴ represents hydrogen or ethoxycarbonyl.

In particularly preferred embodiments, compounds of Formula II for use in the present disclosure are selected from:

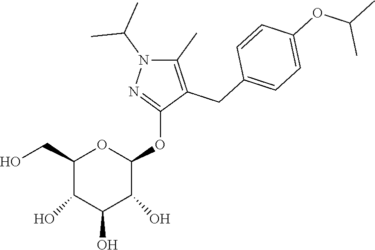 [SMILES: Cc1c(Cc2ccc(OC(C)C)cc2)c(O[C@@H]2O[C@H](CO)[C@@H](O)[C@H](O)[C@H]2O)nn1C(C)C]

### (2R,3S,4 S,5R,6S)-2-(hydroxymethyl)-6-(4-(4-isopropoxybenzyl)-1-isopropyl-5-methyl-1H-pyrazol-3-yloxy)tetrahydro-2H-pyran-3,4,5-trial

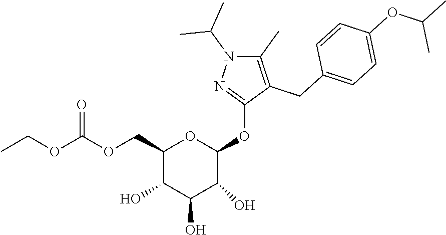 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

### ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate

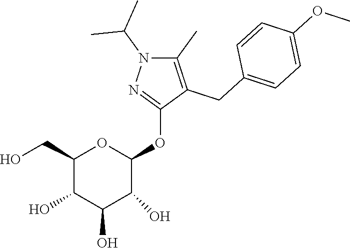 [SMILES: COc1ccc(Cc2c(O[C@@H]3O[C@H](CO)[C@@H](O)[C@H](O)[C@H]3O)nn(C(C)C)c2C)cc1]

### (2R,3S,4S,5R,6S)-2-(hydroxymethyl)-6-(1-isopropyl-4-(4-methoxybenzyl)-5-methyl-1H-pyrazol-3-yloxy)tetrahydro-2H-pyran-3,4,5-triol

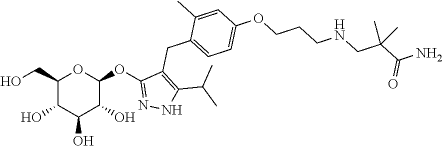 [SMILES: Cc1cc(OCCCNCC(C)(C)C(N)=O)ccc1Cc1c(O[C@@H]2O[C@H](CO)[C@@H](O)[C@H](O)[C@H]2O)n[nH]c1C(C)C]

### 3-((3-(4-((5-isopropyl-3-(((2S,3R,4S,5S,6R)-3,4,5-trihydroxy-6-(hydroxymethyl)tetrahydro-2H-pyran-2-yl)oxy)-1H-pyrazol-4-yl)methyl)-3-methylphenoxy)propyl)amino)-2,2-dimethylpropanamide

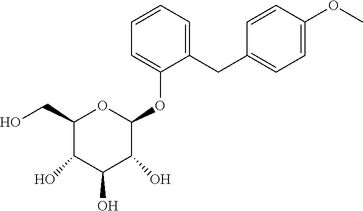 [SMILES: COc1ccc(Cc2ccccc2O[C@@H]2O[C@H](CO)[C@@H](O)[C@H](O)[C@H]2O)cc1]

### (2R,3S,4S,5R,6S)-2-(hydroxymethyl)-6-(2-(4-methoxybenzyl)phenoxy)tetrahydro-2H-pyran-3,4,5-triol

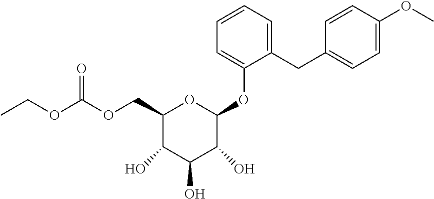 [SMILES: CCOC(=O)OC[C@H]1O[C@@H](Oc2ccccc2Cc2ccc(OC)cc2)[C@H](O)[C@@H](O)[C@@H]1O]

### ethyl ((2R,3S,4S,5R,6S)-3,4,5-trihydroxy-6-(2-(4-methoxybenzyl)phenoxy)tetrahydro-2H-pyran-2-yl)methyl carbonate

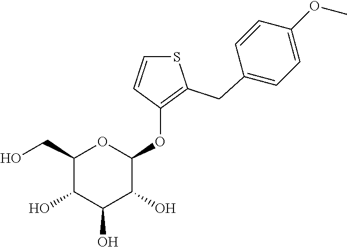 [SMILES: COc1ccc(Cc2sccc2O[C@@H]2O[C@H](CO)[C@@H](O)[C@H](O)[C@H]2O)cc1]

### (2R,3S,4S,5R,6S)-2-(hydroxymethyl)-6-(2-(4-methoxybenzyl)thiophen-3-yloxy)tetrahydro-2H-pyran-3,4,5-triol

### and

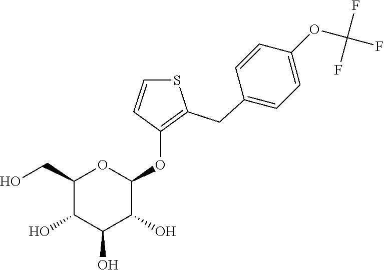 [SMILES: OC[C@H]1O[C@@H](Oc2ccsc2Cc2ccc(OC(F)(F)F)cc2)[C@H](O)[C@@H](O)[C@@H]1O]

### (2R,3S,4S,5R,6S)-2-(hydroxymethyl)-6-(2-(4-(trifluoromethoxy)benzyl)thiophen-3-yloxy)tetrahydro-2H-pyran-3,4,5-triol

In another aspect, the compound that inhibits SGLT for use in the present disclosure are compounds of Formula III:

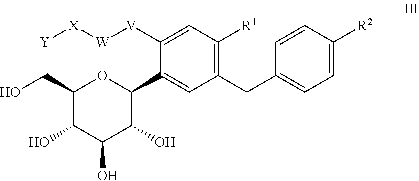

wherein

V represents oxygen or a single bond;

W represents C₁-C₆ alkylene;

X represents oxygen or sulfur;

Y represents C₁-C₆ haloalkyl, C₁-C₆ hydroxyalkyl, C₂-C₆ alkenyl, C₂-C₆ alkynyl, C₃-C₁₀ cycloalkyl, C₅-C₁₀ cycloalkenyl, (C₁-C₄ alkoxy)C₁-C₃ alkyl, (C₂-C₄ alkenyloxy)C₁-C₃ alkyl or (C₃-C₁₀ cycloalkyloxy)C₁-C₃ alkyl;

wherein alkyl, alkenyl, alkynyl, cycloalkyl and cycloalkenyl groups or portions optionally may be partly or completely fluorinated and may be mono- or disubstituted by identical or different substituents selected from chlorine, hydroxy, C₁-C₃ alkyl and C₁-C₃ alkoxy;

R¹ represents hydrogen, halo, cyano, C₁-C₆ alkyl or C₃-C₁₀ cycloalkyl; and

R² represents hydrogen, halo, hydroxy, C₁-C₆ alkyl, C₁-C₆ alkyloxy, C₂-C₆ alkenyl, C₂-C₆ alkynyl, C₃-C₁₀ cycloalkyl, or C₃-C₁₀ cycloalkoxy, wherein alkyl and cycloalkyl groups or portions optionally may be partly or completely fluorinated.

In certain preferred embodiments of compounds of Formula III for use in the disclosure, V represents oxygen or a single bond; W represents C₁-C₃ alkylene; X represents oxygen; Y represents C₁-C₃ haloalkyl, C₂-C₄ alkenyl or C₂-C₄ alkynyl; R¹ represents halo; and R² represents C₁-C₃ alkyl or C₁-C₃ alkoxy.

In particularly preferred embodiments, compounds of Formula III for use in the present disclosure are selected from:

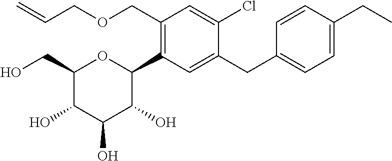 [SMILES: C=CCOCc1cc(Cl)c(Cc2ccc(CC)cc2)cc1[C@@H]1O[C@H](CO)[C@@H](O)[C@H](O)[C@H]1O]

### (2S,3R,4R,5S,6R)-2-(2-(allyloxymethyl)-4-chloro-5-(4-ethylbenzyl)phenyl)-6-(hydroxymethyl)tetrahydro-2H-pyran-3,4,5-triol

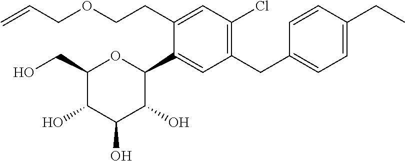 [SMILES: C=CCOCCc1cc(Cl)c(Cc2ccc(CC)cc2)cc1[C@@H]1O[C@H](CO)[C@@H](O)[C@H](O)[C@H]1O]

### (2S,3R,4R,5S,6R)-2-(2-(2-(allyloxy)ethyl)-4-chloro-5-(4-ethylbenzyl)phenyl)-6-(hydroxymethyl)tetrahydro-2H-pyran-3,4,5-triol

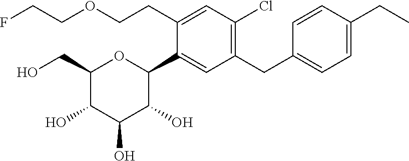 [SMILES: CCc1ccc(Cc2cc([C@@H]3O[C@H](CO)[C@@H](O)[C@H](O)[C@H]3O)c(CCOCCF)cc2Cl)cc1]

### (2S,3R,4R,5S,6R)-2-(4-chloro-5-(4-ethylbenzyl)-2-(2-(2-fluoroethoxy)ethyl)phenyl)-6-(hydroxymethyl)tetrahydro-2H-pyran-3,4,5-triol

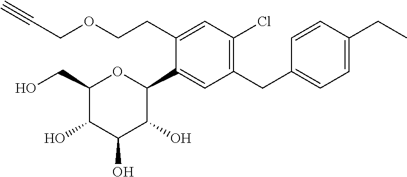 [SMILES: C#CCOCCc1cc(Cl)c(Cc2ccc(CC)cc2)cc1[C@@H]1O[C@H](CO)[C@@H](O)[C@H](O)[C@H]1O]

### (2S,3R,4R,5S,6R)-2-(4-chloro-5-(4-ethylbenzyl)-2-(2-(prop-2-ynyloxy)ethyl)phenyl)-6-(hydroxymethyl)tetrahydro-2H-pyran-3,4,5-triol

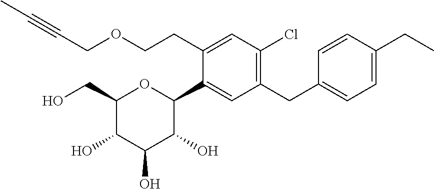 [SMILES: CC#CCOCCc1cc(Cl)c(Cc2ccc(CC)cc2)cc1[C@@H]1O[C@H](CO)[C@@H](O)[C@H](O)[C@H]1O]

### (2S,3R,4R,5S,6R)-2-(2-(2-(but-2-ynyloxy)ethyl)-4-chloro-5-(4-ethylbenzyl)phenyl)-6-(hydroxymethyl)tetrahydro-2H-pyran-3,4,5-triol

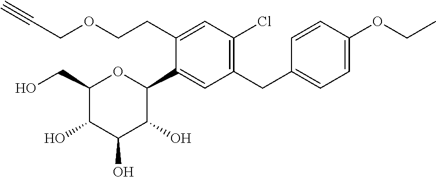 [SMILES: C#CCOCCc1cc(Cl)c(Cc2ccc(OCC)cc2)cc1[C@@H]1O[C@H](CO)[C@@H](O)[C@H](O)[C@H]1O]

### (2S,3R,4R,5S,6R)-2-(4-chloro-5-(4-ethoxybenzyl)-2-(2-(prop-2-ynyloxy)ethyl)phenyl)-6-(hydroxymethyl)tetrahydro-2H-pyran-3,4,5-triol

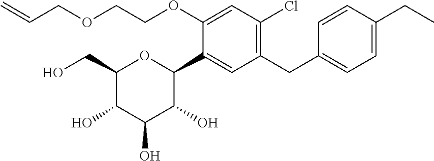 [SMILES: C=CCOCCOc1cc(Cl)c(Cc2ccc(CC)cc2)cc1[C@@H]1O[C@H](CO)[C@@H](O)[C@H](O)[C@H]1O]

### (2S,3R,4R,5S,6R)-2-(2-(2-(allyloxy)ethoxy)-4-chloro-5-(4-ethylbenzyl)phenyl)-6-(hydroxymethyl)tetrahydro-2H-pyran-3,4,5-triol

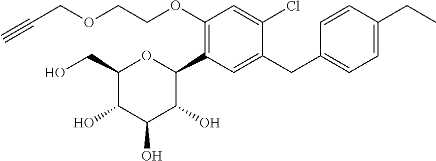 [SMILES: C#CCOCCOc1cc(Cl)c(Cc2ccc(CC)cc2)cc1[C@@H]1O[C@H](CO)[C@@H](O)[C@H](O)[C@H]1O]

### (2S,3R,4R,5S,6R)-2-(4-chloro-5-(4-ethylbenzyl)-2-(2-(prop-2-ynyloxy)ethoxy)phenyl)-6-(hydroxymethyl)tetrahydro-2H-pyran-3,4,5-triol

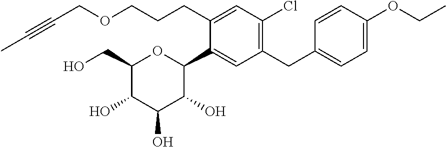 [SMILES: CC#CCOCCCc1cc(Cl)c(Cc2ccc(OCC)cc2)cc1[C@@H]1O[C@H](CO)[C@@H](O)[C@H](O)[C@H]1O]

### (2S,3R,4R,5S,6R)-2-(2-(3-(but-2-ynyloxy)propyl)-4-chloro-5-(4-ethoxybenzyl)phenyl)-6-(hydroxymethyl)tetrahydro-2H-pyran-3,4,5-triol and

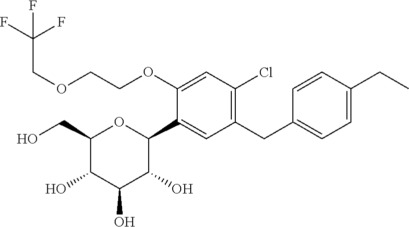 [SMILES: CCc1ccc(Cc2cc([C@@H]3O[C@H](CO)[C@@H](O)[C@H](O)[C@H]3O)c(OCCOCC(F)(F)F)cc2Cl)cc1]

### (2 S,3R,4R,5S,6R)-2-(4-chloro-5-(4-ethylbenzyl)-2-(2-(2,2,2-trifluoroethoxy)ethoxy)phenyl)-6-(hydroxymethyl)tetrahydro-2H-pyran-3,4,5-triol

In another aspect, the compound that inhibits SGLT for use in the present disclosure are compounds of Formula IV:

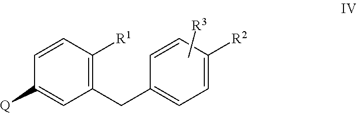

wherein

R¹ and R³ each represent hydrogen, halo, C₁-C₃ alkyl or C₁-C₃ alkoxy; R² represents C₁-C₃ alkyl, C₂-C₄ alkenyl, C₂-C₄ alkynyl, C₃-C₆ cycloalkyl or C₁-C₃ alkoxy; and Q is selected from the following formulae Q^{1A} to Q^{6A}:

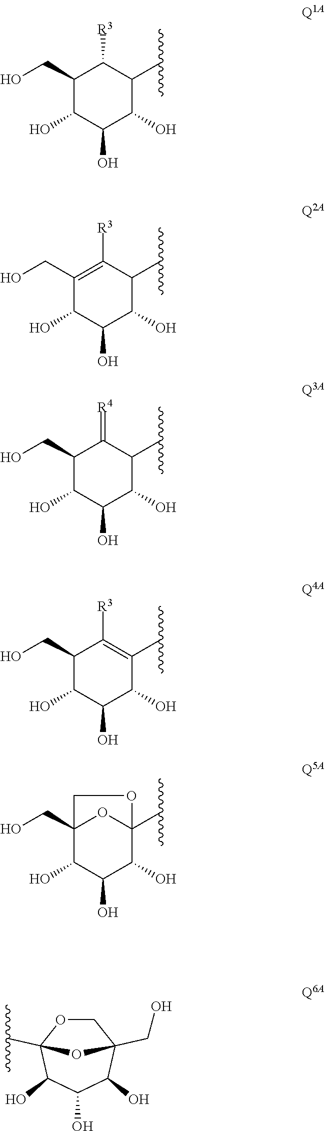

wherein R³ represents hydrogen or hydroxy, and R⁴ represents oxygen or CR^{a}R^{b}, wherein R^{a} and R^{b} each independently represent hydrogen or halo.

In certain preferred embodiments of compounds of Formula IV for use in the disclosure, R¹ represents hydrogen or halo; R² represents C₁-C₃ alkyl or C₁-C₃ alkoxy; R³ represents hydrogen or hydroxy; and R⁴ represents oxygen.

In particularly preferred embodiments, compounds of Formula IV for use in the present disclosure are selected from:

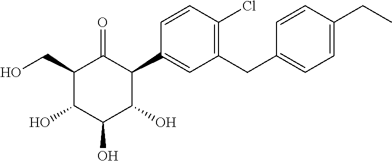 [SMILES: CCc1ccc(Cc2cc([C@@H]3C(=O)[C@H](CO)[C@@H](O)[C@H](O)[C@H]3O)ccc2Cl)cc1]

### (2S,3 S,4R,5R,6R)-2-(4-chloro-3-(4-ethylbenzyl)phenyl)-3,4,5-trihydroxy-6-(hydroxymethyl)cyclohexanone

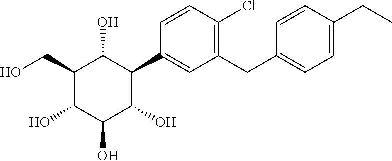 [SMILES: CCc1ccc(Cc2cc([C@H]3[C@H](O)[C@@H](O)[C@H](O)[C@@H](CO)[C@@H]3O)ccc2Cl)cc1]

### (1R,2R,3S,4R,5R,6S)-4-(4-chloro-3-(4-ethylbenzyl)phenyl)-6-(hydroxymethyl)cyclohexane-1,2,3,5-tetraol

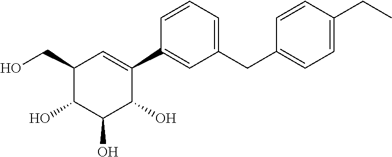

### (1R,2S,3S,6R)-4-(3-(4-ethylbenzyl)phenyl)-6-(hydroxymethyl)cyclohex-4-ene-1,2,3-triol

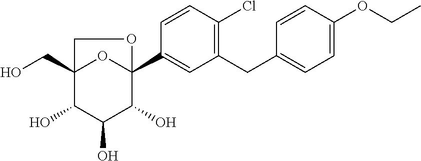 [SMILES: CCOc1ccc(Cc2cc([C@]34OC[C@](CO)(O3)[C@@H](O)[C@H](O)[C@H]4O)ccc2Cl)cc1]

### (1 S,2S,3S,4R,5S)-5-(4-chloro-3-(4-ethoxybenzyl)phenyl)-1-(hydroxymethyl)-6,8-dioxabicyclo[3.2.1]octane-2,3,4-triol

### and

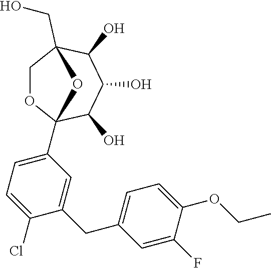 [SMILES: CCOc1ccc(Cc2cc([C@@]34OC[C@@](CO)(O3)[C@@H](O)[C@H](O)[C@H]4O)ccc2Cl)cc1F]

### (1R,2S,3S,4R,5R)-5-[4-chloro-3-[(4-ethoxy-3-fluorophenyl)methyl]phenyl]-1-(hydroxymethyl)-6,8-dioxabicyclo[3.2.1]octane-2,3,4-triol

In another aspect, the compound that inhibits SGLT for use in the present disclosure are compounds of Formula V:

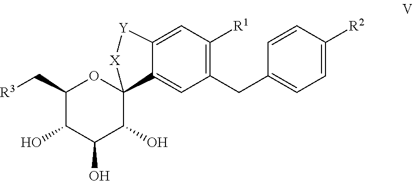

wherein

X represents methylene or oxygen; Y represents (CH₂)ₙ, (CH₂)ₘCH═CH, CH═CH(CH₂)ₘ or CH₂CH═CHCH₂, wherein n is an integer from 1 to 3 and m is an integer from 0 to 2; R¹ represents hydrogen or halo; R² represents hydrogen, halo, C₁-C₃ alkyl, C₂-C₄ alkynyl, C₃-C₆ cycloalkyl, C₁-C₃ alkoxy, C₃-C₆ cycloalkoxy, hydroxy or cyano; R³ represents hydroxy, fluoro or C₁-C₃ alkoxy; and wherein alkyl groups or portions optionally may be partly or completely fluorinated.

In certain preferred embodiments of compounds of Formula V for use in the disclosure, X represents oxygen; Y represents CH₂; R¹ represents hydrogen or halo; R² represents C₁-C₃ alkyl or C₁-C₃ alkoxy, wherein the alkyl group or portion optionally may be partly or completely fluorinated; and R³ represents hydroxy.

In other preferred embodiments of compounds of Formula V for use in the disclosure, X represents methylene; Y represents CH₂; R¹ represents hydrogen or halo; R² represents C₁-C₃ alkyl or C₁-C₃ alkoxy, wherein the alkyl group or portion optionally may be partly or completely fluorinated; and R³ represents hydroxy.

In particularly preferred embodiments, compounds of Formula V for use in the present disclosure are selected from:

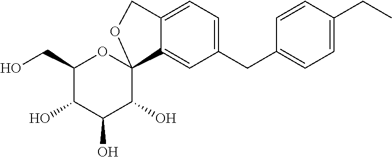 [SMILES: CCc1ccc(Cc2ccc3c(c2)[C@]2(OC3)O[C@H](CO)[C@@H](O)[C@H](O)[C@H]2O)cc1]

### (1S,3′R,4'S,5'S,6′R)-6-(4-ethylbenzyl)-6′-(hydroxymethyl)-3′,4′,5′,6′-tetrahydro-3H-spiro[isobenzofuran-1,2′-pyran]-3′,4′,5′-triol

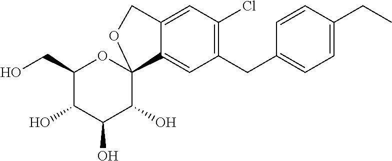 [SMILES: CCc1ccc(Cc2cc3c(cc2Cl)CO[C@]32O[C@H](CO)[C@@H](O)[C@H](O)[C@H]2O)cc1]

### (1 S,3′R,4'S,5'S,6′R)-5-chloro-6-(4-ethylbenzyl)-6′-(hydroxymethyl)-3′,4′,5′,6′-tetrahydro-3H-spiro[isobenzofuran-1,2′-pyran]-3′,4′,5′-triol

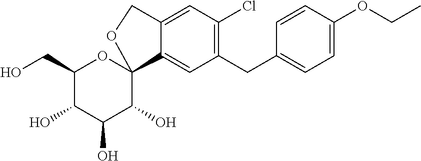 [SMILES: CCOc1ccc(Cc2cc3c(cc2Cl)CO[C@]32O[C@H](CO)[C@@H](O)[C@H](O)[C@H]2O)cc1]

### (1 S,3′R,4'S,5'S,6′R)-5-chloro-6-(4-ethoxybenzyl)-6′-(hydroxymethyl)-3′,4′,5′,6′-tetrahydro-3H-spiro[isobenzofuran-1,2′-pyran]-3′,4′,5′-triol

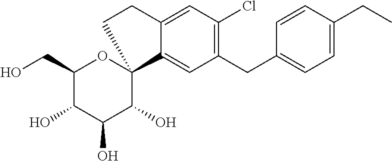 [SMILES: CCc1ccc(Cc2cc3c(cc2Cl)CC[C@]32O[C@H](CO)[C@@H](O)[C@H](O)[C@H]2O)cc1]

### (1 S,3′R,4'S,5'S,6′R)-5-chloro-6-(4-ethylbenzyl)-6′-(hydroxymethyl)-2,3,3′,4′,5′,6′-hexahydrospiro[indene-1,2′-pyran]-3′,4′,5′-triol

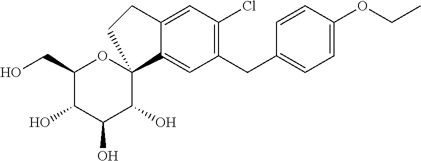 [SMILES: CCOc1ccc(Cc2cc3c(cc2Cl)CC[C@]32O[C@H](CO)[C@@H](O)[C@H](O)[C@H]2O)cc1]

### (1 S,3′R,4'S,5'S,6′R)-5-chloro-6-(4-ethoxybenzyl)-6′-(hydroxymethyl)-2,3,3′,4′,5′,6′-hexahydrospiro[indene-1,2′-pyran]-3′,4′,5′-triol

### and

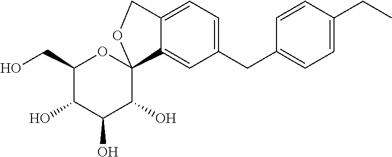 [SMILES: CCc1ccc(Cc2ccc3c(c2)[C@]2(OC3)O[C@H](CO)[C@@H](O)[C@H](O)[C@H]2O)cc1]

### (1S,3′R,4'S,5'S,6′R)-6-(4-ethylbenzyl)-6′-(hydroxymethyl)-3′,4′,5′,6′-tetrahydro-3H-spiro[isobenzofuran-1,2′-pyran]-3′,4′,5′-triol

The present disclosure includes the use of all tautomers and stereoisomers of the afore-mentioned compounds, either in admixture or in pure or substantially pure form. The compounds can have asymmetric centers at the carbon atoms, and therefore the compounds can exist in diastereomeric or enantiomeric forms or mixtures thereof. The use of all conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, co-crystals and tautomers are within the scope of the present disclosure. The compounds can be prepared using diastereomers, enantiomers or racemic mixtures as starting materials. Furthermore, diastereomer and enantiomer products can be separated by chromatography, fractional crystallization or other methods known to those of skill in the art.

The present disclosure also provides for the use of prodrugs of the afore-mentioned compounds. Prodrugs of the compounds include, but are not limited to, carboxylate esters, carbonate esters, hemi-esters, phosphorus esters, nitro esters, sulfate esters, sulfoxides, amides, carbamates, azo compounds, phosphamides, glycosides, ethers, acetals, and ketals. Prodrug esters and carbonates may be formed, for example, by reacting one or more hydroxyl groups of the compounds with alkyl, alkoxy or aryl substituted acylating reagents using methods known to those of skill in the art to produce methyl carbonates, acetates, benzoates, pivalates and the like. Illustrative examples of prodrug esters of the compounds include, but are not limited to, compounds having a hydroxy moiety wherein the free hydrogen is replaced by (C₁-C₆ alkyl)oxycarbonyl, (C₁-C₆ alkyl)carbonyl, phenyloxycarbonyl, benzylcarbonyl or benzyloxycarbonyl. The use of oligopeptide modifications and biodegradable polymer derivatives (as described, for example, in Int. J. Pharm. 115, 61-67, 1995) are within the scope of the disclosure. Methods for selecting and preparing suitable prodrugs are provided, for example, in the following: T. Higuchi and V. Stella, “Prodrugs as Novel Delivery Systems,” Vol. 14, ACS Symposium Series, 1975; H. Bundgaard, “Design of Prodrugs,” Elsevier, 1985; and “Bioreversible Carriers in Drug Design,” ed. Edward Roche, American Pharmaceutical Association and Pergamon Press, 1987.

In certain instances, the above-referenced compounds are in prodrug form. For example:

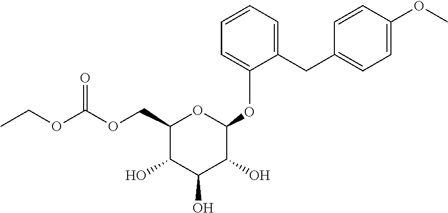 [SMILES: CCOC(=O)OC[C@H]1O[C@@H](Oc2ccccc2Cc2ccc(OC)cc2)[C@H](O)[C@@H](O)[C@@H]1O]

### ethyl ((2R,3S,4S,5R,6S)-3,4,5-trihydroxy-6-(2-(4-methoxybenzyl)phenoxy)tetrahydro-2H-pyran-2-yl)methyl carbonate

is a prodrug of

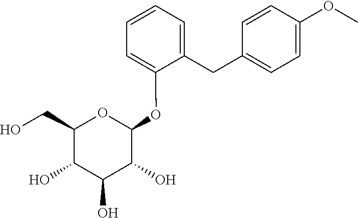 [SMILES: COc1ccc(Cc2ccccc2O[C@@H]2O[C@H](CO)[C@@H](O)[C@H](O)[C@H]2O)cc1]

### (2R,3S,4S,5R,6S)-2-(hydroxymethyl)-6-(2-(4-methoxybenzyl)phenoxy)tetrahydro-2H-pyran-3,4,5-triol, and

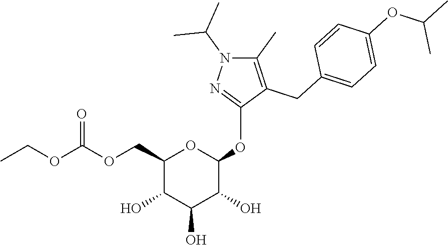 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

### ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate

is a prodrug of

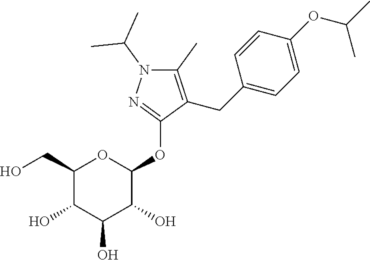 [SMILES: Cc1c(Cc2ccc(OC(C)C)cc2)c(O[C@@H]2O[C@H](CO)[C@@H](O)[C@H](O)[C@H]2O)nn1C(C)C]

### (2R,3S,4 S,5R,6S)-2-(hydroxymethyl)-6-(4-(4-isopropoxybenzyl)-1-isopropyl-5-methyl-1H-pyrazol-3-yloxy)tetrahydro-2H-pyran-3,4,5-triol

In some embodiments, the compound that inhibits an SGLT or the prodrug thereof has a structure selected from the group consisting of

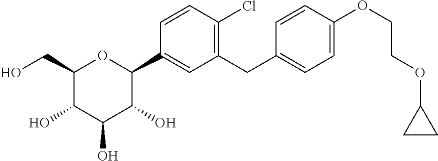 [SMILES: OC[C@H]1O[C@@H](c2ccc(Cl)c(Cc3ccc(OCCOC4CC4)cc3)c2)[C@H](O)[C@@H](O)[C@@H]1O]

### (2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol

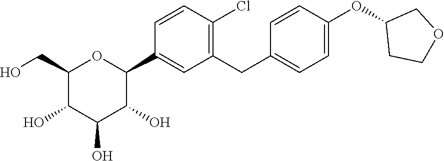 [SMILES: OC[C@H]1O[C@@H](c2ccc(Cl)c(Cc3ccc(O[C@H]4CCOC4)cc3)c2)[C@H](O)[C@@H](O)[C@@H]1O]

### (2S,3R,4R,5S,6R)-2-(4-chloro-3-(4-((S)-tetrahydrofuran-3-yloxy)benzyl)phenyl)-6-(hydroxymethyl)tetrahydro-2H-pyran-3,4,5-triol

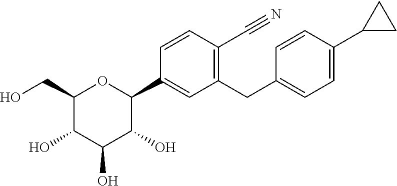 [SMILES: N#Cc1ccc([C@@H]2O[C@H](CO)[C@@H](O)[C@H](O)[C@H]2O)cc1Cc1ccc(C2CC2)cc1]

### 2-(4-cyclopropylbenzyl)-4-((2S,3R,4R,5S,6R)-3,4,5,-trihydroxy-6-(hydroxymethyl)tetrahydro-2H-pyran-2-yl)benzonitrile

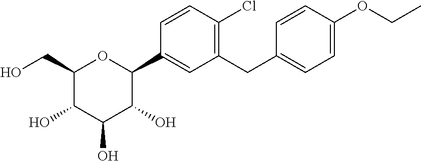 [SMILES: CCOc1ccc(Cc2cc([C@@H]3O[C@H](CO)[C@@H](O)[C@H](O)[C@H]3O)ccc2Cl)cc1]

### (2S,3R,4R,5S,6R)-2-(4-chloro-3-(4-ethoxybenzyl)phenyl)-6-(hydroxymethyl)tetrahydro-2H-pyran-3,4,5-triol

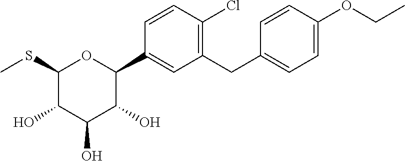 [SMILES: CCOc1ccc(Cc2cc([C@@H]3O[C@H](SC)[C@@H](O)[C@H](O)[C@H]3O)ccc2Cl)cc1]

### (2S,3R,4R,5S,6R)-2-(4-chloro-3-(4-ethoxybenzyl)phenyl)-6-(methylthio)tetrahydro-2H-pyran-3,4,5-triol

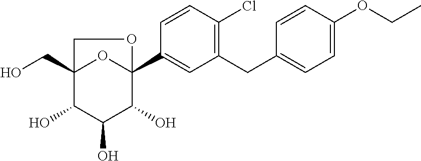 [SMILES: CCOc1ccc(Cc2cc([C@]34OC[C@](CO)(O3)[C@@H](O)[C@H](O)[C@H]4O)ccc2Cl)cc1]

### (1S,2S,3S,4R,5S)-5-(4-chloro-3-(4-ethoxybenzyl)phenyl)-1-(hydroxymethyl)-6,8-dioxabicyclo[3.2.1]octane-2,3,4-triol

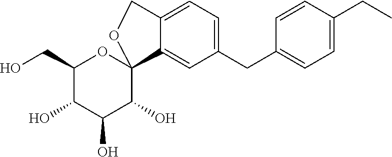 [SMILES: CCc1ccc(Cc2ccc3c(c2)[C@]2(OC3)O[C@H](CO)[C@@H](O)[C@H](O)[C@H]2O)cc1]

### (1S,3′R,4'S,5'S,6′R)-6-(4-ethylbenzyl)-6′-(hydroxymethyl)-3′,4′,5′,6′-tetrahydro-3H-spiro[isobenzofuran-1,2′-pyran]-3′,4′,5′-triol

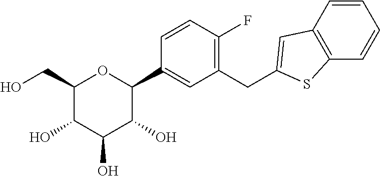 [SMILES: OC[C@H]1O[C@@H](c2ccc(F)c(Cc3cc4ccccc4s3)c2)[C@H](O)[C@@H](O)[C@@H]1O]

### (2 S,3R,4R,5S,6R)-2-(3-(benzo[b]thio phe n-2-ylmethyl)-4-fluorophenyl)-6-(hydroxymethyl)tetrahydro-2H-pyran-3,4,5-triol

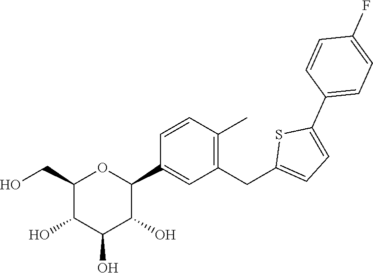 [SMILES: Cc1ccc([C@@H]2O[C@H](CO)[C@@H](O)[C@H](O)[C@H]2O)cc1Cc1ccc(-c2ccc(F)cc2)s1]

### (2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol

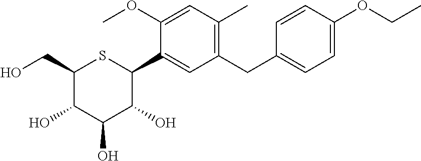 [SMILES: CCOc1ccc(Cc2cc([C@@H]3S[C@H](CO)[C@@H](O)[C@H](O)[C@H]3O)c(OC)cc2C)cc1]

### (2 S,3R,4R,5S,6R)-2-(5-(4-ethoxybenzyl)-2-methoxy-4-methylphenyl)-6-(hydroxymethyl)tetrahydro-2H-thiopyran-3,4,5-triol

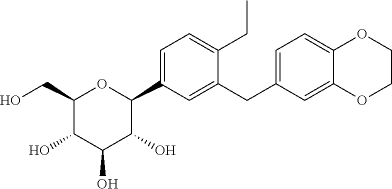 [SMILES: CCc1ccc([C@@H]2O[C@H](CO)[C@@H](O)[C@H](O)[C@H]2O)cc1Cc1ccc2c(c1)OCCO2]

### (2S,3R,4R,5S,6R)-2-[3-(2,3-Dihydro-benzo[1,4]dioxin-6-ylmethyl)-4-ethyl-phenyl]-6-hydroxymethyl-tetrahydro-pyran-3,4,5-triol

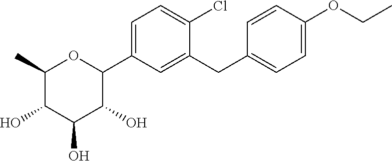 [SMILES: CCOc1ccc(Cc2cc(C3O[C@H](C)[C@@H](O)[C@H](O)[C@H]3O)ccc2Cl)cc1]

### (3R,4S,5S,6R)-2-(4-chloro-3-(4-ethoxybenzyl)phenyl)-6-methyltetrahydro-2H-pyran-3,4,5-triol

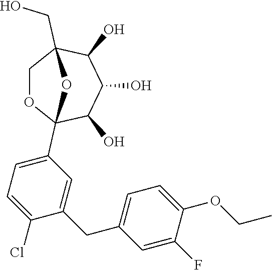 [SMILES: CCOc1ccc(Cc2cc([C@@]34OC[C@@](CO)(O3)[C@@H](O)[C@H](O)[C@H]4O)ccc2Cl)cc1F]

### (1R,2S,3S,4R,5R)-5-[4-chloro-3-[(4-ethoxy-3-fluorophenyl)methyl]phenyl]-1-(hydroxymethyl)-6,8-dioxabicyclo[3.2.1]octane-2,3,4-triol

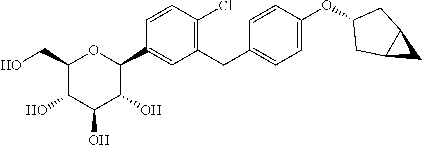 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]

### (2S,3R, 4R, 5S, 6R)-2-(3-(4-(((1R, 3s, 5S)-bicyclo[3.1.0]hexan-3-yl)oxy)benzyl)-4-chlorophenyl)-6-(hydroxymethyl)tetrahydro-2H-pyran-3,4,5-triol

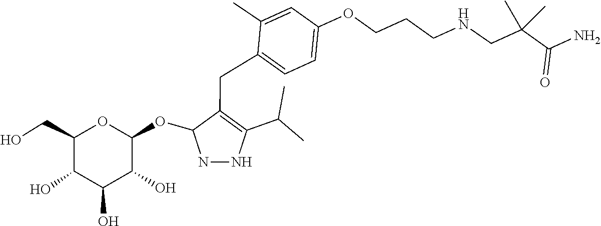

### 3-((3-(4-((5-isopropyl-3-(((2 S, 3R, 4S, 5S,6R)-3,4,5-trihydroxy-6-(hydroxymethyl)tetrahydro-2H-pyran-2-yl)oxy)-1H-pyrazol-4-yl)methyl)-3-methylphenoxy)propyl)amino)-2,2-dimethylpropanamide

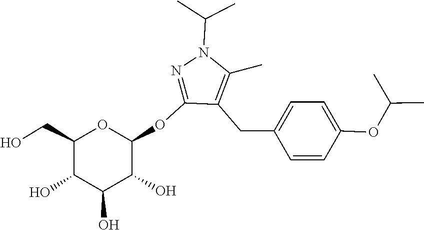 [SMILES: Cc1c(Cc2ccc(OC(C)C)cc2)c(O[C@@H]2O[C@H](CO)[C@@H](O)[C@H](O)[C@H]2O)nn1C(C)C]

### (2R,3S,4S,5R,6S)-2-(hydroxymethyl)-6-((4-(4-isopropoxybenzyl)-1-isopropyl-5-methyl-1H-pyrazol-3-yl)oxy)tetrahydro-2H-pyran-3,4,5-triol

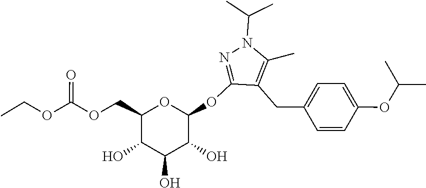 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

### ethyl (((2R,3S,4S,5R,6S)-3,4,5-trihydroxy-6-((4-(4-isopropoxybenzyl)-1-isopropyl-5-methyl-1H-pyrazol-3-yl)oxy)tetrahydro-2H-pyran-2-yl)methyl) carbonate

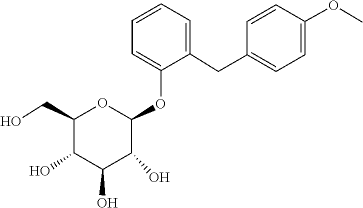 [SMILES: COc1ccc(Cc2ccccc2O[C@@H]2O[C@H](CO)[C@@H](O)[C@H](O)[C@H]2O)cc1]

### (2R,3S,4S,5R,6S)-2-(hydroxymethyl)-6-(2-(4-methoxybenzyl)phenoxy)tetrahydro-2H-pyran-3,4,5-triol

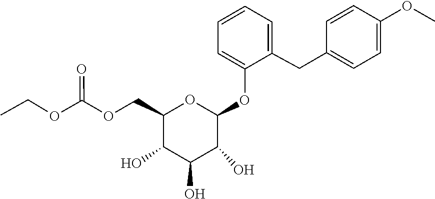 [SMILES: CCOC(=O)OC[C@H]1O[C@@H](Oc2ccccc2Cc2ccc(OC)cc2)[C@H](O)[C@@H](O)[C@@H]1O]

### ethyl (((2R,3S,4S,5R,6S)-3,4,5-trihydroxy-6-(2-(4-methoxybenzyl)phenoxy)tetrahydro-2H-pyran-2-yl)methyl) carbonate

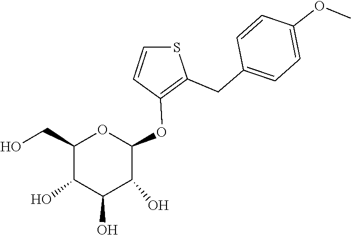 [SMILES: COc1ccc(Cc2sccc2O[C@@H]2O[C@H](CO)[C@@H](O)[C@H](O)[C@H]2O)cc1]

### (2R,3 S,4 S,5R,6 S)-2-(hydroxymethyl)-6-(2-(4-methoxybenzyl)thiophen-3-yloxy)tetrahydro-2H-pyran-3,4,5-triol and

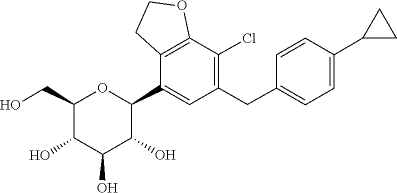 [SMILES: OC[C@H]1O[C@@H](c2cc(Cc3ccc(C4CC4)cc3)c(Cl)c3c2CCO3)[C@H](O)[C@@H](O)[C@@H]1O]

### (2S,3R,4R,5S,6R)-2-(7-chloro-6-(4-cyclopropylbenzyl)-2,3-dihydrobenzofuran-4-yl)-6-(hydroxymethyl)tetrahydro-2H-pyran-3,4,5-triol

In some embodiments, bexagliflozin is used as the compound that inhibits a sodium-dependent glucose transporter (SGLT).

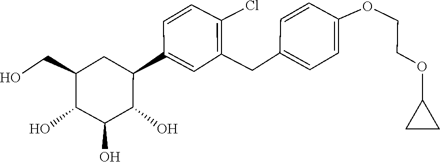 [SMILES: OC[C@H]1C[C@@H](c2ccc(Cl)c(Cc3ccc(OCCOC4CC4)cc3)c2)[C@H](O)[C@@H](O)[C@@H]1O]

### (2S,3R,4R,5 S,6R)-2-(4-chloro-3-(4-(2-cyclopropoxyethoxy)benzyl)phenyl)-6-(hydroxymethyl)tetrahydro-2H-pyran-3,4,5-triol

Bexagliflozin is a highly effective and specific inhibitor of human SGLT2 and a potent but less selective inhibitor of feline SGLT2. In particular, bexagliflozin has high potency for feline SGLT1.

In some embodiments, the compound that inhibits an SGLT selectively inhibits SGLT1. In some embodiments, the compound that inhibits an SGLT selectively inhibits SGLT2.

B. Methods of Treating Heart Failure

In some aspects, provided herein are methods for the treatment of heart failure in a companion animal, comprising administering to a subject in need thereof a therapeutically effective amount of a compound that inhibits a sodium-dependent glucose transporter (SGLT) or a prodrug thereof.

Suitable compounds that inhibit a sodium-dependent glucose transporter (SGLT) or a prodrug thereof are described in sub-section A, above.

The companion animals of the described heart failure treatment methods may be preselected based one or more clinical, behavioral, and/or physiological criteria. In some embodiments, the companion animal is preselected to be obese.

In some embodiments, the companion animals are preselected based on the presence or absence of particular conditions or disease states. In some embodiments, the companion animal is preselected to not have type 1 or 2 diabetes. In some embodiments, the companion animal is preselected to not have type 2 diabetes. In some embodiments, the companion animal is preselected to not have chronic kidney disease. In some embodiments, the companion animal is preselected to have chronic kidney disease. In some embodiments, the companion animal is preselected to not be hypertensive. In some embodiments, the companion animal is preselected to be hypertensive.

The methods described herein are suitable for companion animals as described in sub-section E, below.

In some embodiments the companion animal with heart failure has hypertrophic cardiomyopathy. In some embodiments, the companion animal with heart failure has a valvular heart disease. In some embodiments, the valvular heart disease is mitral valve disease. In some embodiments, the valvular heart diseases is aortic valve disease.

In some embodiments, administration of a therapeutically effective amount of a compound that inhibits an SGLT prevents the progression of heart failure such that the New York Heart Association (NYHA) functional classification of the companion animal does not change. In some embodiments, administration of a therapeutically effective amount of a compound that inhibits an SGLT improves the NYHA functional classification of the companion animal.

The treatments described herein can be administered prophylactically to prevent or delay the onset or progression of heart failure, or therapeutically to relieve the symptoms of heart failure for a sustained period of time

C. Methods of Treating Chronic Kidney Disease (CKD)

In some aspects, provided herein are methods for the treatment of chronic kidney disease (CKD) in a companion animal, comprising administering to a subject in need thereof a therapeutically effective amount of a compound that inhibits a sodium-dependent glucose transporter (SGLT) or a prodrug thereof.

Suitable compounds that inhibit a sodium-dependent glucose transporter (SGLT) or a prodrug thereof are described in sub-section A, above.

The companion animals of the described chronic kidney disease treatment methods may be preselected based one or more clinical, behavioral, and/or physiological criteria. In some embodiments, the companion animal is preselected to be obese.

In some embodiments, the companion animals are preselected based on the presence or absence of particular conditions or disease states. In some embodiments, the companion animal is preselected to not have type 1 or 2 diabetes. In some embodiments, the companion animal is preselected to not have type 2 diabetes. In some embodiments, the companion animal is preselected to not have heart failure. In some embodiments, the companion animal is preselected to have heart failure. In some embodiments, the companion animal is preselected to not be hypertensive. In some embodiments, the companion animal is preselected to be hypertensive.

The methods described herein are suitable for companion animals as described in sub-section E, below.

Said treating can increase the amount of blood creatinine in the companion animal. In some embodiments, the increase in blood creatinine in the companion animal is about a 5% increase over the blood creatinine level before treatment. In some embodiments, the increase in blood creatinine in the companion animal is about a 10% increase over the blood creatinine level before treatment. In some embodiments, the increase in blood creatinine in the companion animal is about a 15% increase over the blood creatinine level before treatment.

Said treating can decrease the amount of blood urea nitrogen (BUN) in the companion animal. In some embodiments, the decrease in BUN in the companion animal is about a 5% decrease below the BUN level before treatment. In some embodiments, the decrease in BUN in the companion animal is about a 10% decrease below the BUN level before treatment. In some embodiments, the decrease in BUN in the companion animal is about a 15% decrease below the BUN level before treatment.

Said treating can decrease the amount of symmetric dimethylarginine (SDMA) in the blood of the companion animal. In some embodiments, the decrease in blood SDMA in the companion animal is about a 5% decrease below the blood SDMA level before treatment. In some embodiments, the decrease in blood SDMA in the companion animal is about a 10% decrease below the blood SDMA level before treatment. In some embodiments, the decrease in blood SDMA in the companion animal is about a 15% decrease below the blood SDMA level before treatment.

The observed clinical changes can occur shortly after treatment or after a given time period. The specific amount of time to observe the described clinical changes will vary depending on a number of factors including the companion animal being treated, the dosage amount, and the disease state of the animal. In some embodiments, the comparison in the preceding paragraphs is the change from before treatment to the end of Week 2 of treatment. In some embodiments, the comparison in the preceding paragraphs is the change from before treatment to the end of Week 4 of treatment. In some embodiments, the comparison in the preceding paragraphs is the change from before treatment to the end of Week 8 of treatment. In some embodiments, the comparison in the preceding paragraphs is the change from before treatment to the end of Week 12 of treatment. In some embodiments, the treatment methods described herein provide a durable response.

D. Methods of Treating Hypertension

Also provided herein are methods for the treatment of hypertension in a companion animal, comprising administering to a subject in need thereof a therapeutically effective amount of a compound that inhibits a sodium-dependent glucose transporter (SGLT) or a prodrug thereof.

Suitable compounds that inhibit a sodium-dependent glucose transporter (SGLT) or a prodrug thereof are described in sub-section A, above.

The companion animals of the described hypertension treatment methods may be preselected based one or more clinical, behavioral, and/or physiological criteria. In some embodiments, the companion animal is preselected to be obese.

In some embodiments, the companion animals are preselected based on the presence or absence of particular conditions or disease states. In some embodiments, the companion animal is preselected to not have type 1 or 2 diabetes. In some embodiments, the companion animal is preselected to not have type 2 diabetes. In some embodiments, the companion animal is preselected to not have heart failure. In some embodiments, the companion animal is preselected to have heart failure. In some embodiments, the companion animal is preselected to not have chronic kidney disease. In some embodiments, the companion animal is preselected to have chronic kidney disease.

The methods described herein are suitable for companion animals as described in sub-section E, below.

In some embodiments, said treating reduces resting systolic blood pressure. In some embodiments, said treating reduces resting systolic blood pressure in said companion animal by about 3 to 20 mm Hg. In some embodiments, said treating reduces resting systolic blood pressure in said companion animal by at least 3 mm Hg. In some embodiments, said treating reduces resting systolic blood pressure in said companion animal by at least 5 mm Hg. In some embodiments, said treating reduces resting systolic blood pressure in said companion animal by at least 7 mm Hg. In some embodiments, said treating reduces resting systolic blood pressure in said companion animal by at least 10 mm Hg. In some embodiments, said treating reduces resting systolic blood pressure in said companion animal by at least 15 mm Hg.

In some embodiments, said treating reduces resting diastolic blood pressure. In some embodiments, said treating reduces resting diastolic blood pressure in said companion animal by about 2 to 15 mm Hg. In some embodiments, said treating reduces resting diastolic blood pressure in said companion animal by at least 2 mm Hg. In some embodiments, said treating reduces resting diastolic blood pressure in said companion animal by at least 4 mm Hg. In some embodiments, said treating reduces resting diastolic blood pressure in said companion animal by at least 6 mm Hg. In some embodiments, said treating reduces resting diastolic blood pressure in said companion animal by at least 8 mm Hg. In some embodiments, said treating reduces resting diastolic blood pressure in said companion animal by at least 10 mm Hg.

In some embodiments said treating reduces the pulse pressure, that is, the difference between the systolic and diastolic blood pressures, in said companion animal by about 2 to 15 mm Hg. In some embodiments, said treating reduces resting pulse pressure in said companion animal by at least 2 mm Hg. In some embodiments, said treating reduces resting pulse pressure in said companion animal by at least 5 mm Hg. In some embodiments, said treating reduces resting pulse pressure in said companion animal by at least 7 mm Hg. In some embodiments, said treating reduces resting pulse blood pressure in said companion animal by at least 10 mm Hg.

The observed clinical changes can occur shortly after treatment or after a given time period. The specific amount of time to observe the described clinical changes will vary depending on a number of factors including the companion animal being treated, the dosage amount, and the disease state of the animal. In some embodiments, the comparison in the preceding paragraphs is the change from before treatment to the end of Week 2 of treatment. In some embodiments, the comparison in the preceding paragraphs is the change from before treatment to the end of Week 4 of treatment. In some embodiments, the comparison in the preceding paragraphs is the change from before treatment to the end of Week 8 of treatment. In some embodiments, the comparison in the preceding paragraphs is the change from before treatment to the end of Week 12 of treatment. In some embodiments, the treatment methods described herein provide a durable response.

E. Companion Animals

Companion animals include domestic animals preferably including (for example) canines (dogs), felines (cats), equidae (horses), suidae (pigs), leporidae (rabbits), and the like. In some embodiments, the companion animal is a canine. In some embodiments, the companion animal is a feline.

It is understood that the therapeutically effective amount as well as the frequency of dosing depends on a number of factors including the disease being treated, the severity of the disease, the compound that inhibits a sodium-dependent glucose transporter (SGLT), the route of administration, the companion animal receiving treatment, as well as the size of the companion animal. The dosage can be increased or decreased over time, as required by a given companion animal.

In some embodiments, the therapeutically effective amount administered to a canine is a total daily dosage of about 10-4,000 μg kg⁻¹ of the compound that inhibits an SGLT or the prodrug thereof. In some embodiments, the therapeutically effective amount administered to a canine is a total daily dosage of about 50-3,200 μg kg⁻¹ of the compound that inhibits an SGLT or the prodrug thereof. In some embodiments, the therapeutically effective amount administered to a canine is a total daily dosage selected from the group consisting of about 50 μg kg⁻¹, 100 μg kg⁻¹, 200 μg kg⁻¹, 400 μg kg⁻¹, 800 μg kg⁻¹, 1,000 μg kg⁻¹, 1,600 μg kg⁻¹, and 3,200 μg kg⁻¹ of the compound that inhibits an SGLT or the prodrug thereof. In some embodiments, the therapeutically effective amount administered to a canine is a total daily dosage of about 1,000 μg kg⁻¹ of the compound that inhibits an SGLT or the prodrug thereof.

In some embodiments, the therapeutically effective amount administered to a canine is a total daily dosage of about 100-40,000 μg kg⁻¹ of the compound that inhibits an SGLT or the prodrug thereof. In some embodiments, the therapeutically effective amount administered to a canine is a total daily dosage of about 500-3,2000 μg kg⁻¹ of the compound that inhibits an SGLT or the prodrug thereof. In some embodiments, the therapeutically effective amount administered to a canine is a total daily dosage selected from the group consisting of about 500 μg kg⁻¹, 1,000 μg kg⁻¹, 2,000 μg kg⁻¹, 4,000 μg kg⁻¹, 8,000 μg kg⁻¹, 10,000 μg kg⁻¹, 16,000 μg kg⁻¹, and 32,000 μg kg⁻¹ of the compound that inhibits an SGLT or the prodrug thereof. In some embodiments, the therapeutically effective amount administered to a canine is a total daily dosage of about 10,000 μg kg⁻¹ of the compound that inhibits an SGLT or the prodrug thereof. A person of skill in the art will appreciate that less potent compounds that inhibit an SGLT will require higher dosages. Less potent compounds include, for example, certain SGLT inhibitors such as O-glycosides.

In some embodiments, the above-referenced amounts are suitable for treating canines with heart failure. In some embodiments, the above-referenced amounts are suitable for treating canines with CKD. In some embodiments, the above-referenced amounts are suitable for treating canines with hypertension.

In some embodiments, the therapeutically effective amount administered to a feline is a total daily dosage of about 100-30,000 μg kg⁻¹ of the compound that inhibits an SGLT or the prodrug thereof. In some embodiments, the therapeutically effective amount administered to a feline is a total daily dosage of about 200-25,600 μg kg⁻¹ of the compound that inhibits an SGLT or the prodrug thereof. In some embodiments, the therapeutically effective amount administered to a feline is a total daily dosage selected from the group consisting of about 200 μg kg⁻¹, 400 μg kg⁻¹, 800 μg kg⁻¹, 1,000 μg kg⁻¹, 1,600 μg kg⁻¹, 2,500 μg kg⁻¹, 3,200 μg kg⁻¹, 5,000 μg kg⁻¹, 6,400 μg kg⁻¹, 12,800 μg kg⁻¹, and 25,600 of the compound that inhibits an SGLT or the prodrug thereof. In some embodiments, the therapeutically effective amount administered to a feline is a total daily dosage of about 2,500 μg kg⁻¹ of the compound that inhibits an SGLT or the prodrug thereof. In some embodiments, the therapeutically effective amount administered to a feline is a total daily dosage of about 5,000 μg kg⁻¹ g kg⁻¹ of the compound that inhibits an SGLT or the prodrug thereof.

In some embodiments, the therapeutically effective amount administered to a feline is a total daily dosage of about 1,000-300,000 μg kg⁻¹ of the compound that inhibits an SGLT or the prodrug thereof. In some embodiments, the therapeutically effective amount administered to a feline is a total daily dosage of about 2,000-256,000 μg kg⁻¹ of the compound that inhibits an SGLT or the prodrug thereof. In some embodiments, the therapeutically effective amount administered to a feline is a total daily dosage selected from the group consisting of about 2,000 μg kg⁻¹, 4,000 μg kg⁻¹, 8,000 μg kg⁻¹, 10,000 μg kg⁻¹, 16,000 μg kg⁻¹, 25,000 μg kg⁻¹, 32,000 μg kg⁻¹, 50,000 μg kg⁻¹, 64,000 μg kg⁻¹, 128,000 μg kg⁻¹, and 256,000 of the compound that inhibits an SGLT or the prodrug thereof. In some embodiments, the therapeutically effective amount administered to a feline is a total daily dosage of about 25,000 μg kg⁻¹ of the compound that inhibits an SGLT or the prodrug thereof. In some embodiments, the therapeutically effective amount administered to a feline is a total daily dosage of about 50,000 μg kg⁻¹ μg kg⁻¹ of the compound that inhibits an SGLT or the prodrug thereof. A person of skill in the art will appreciate that less potent compounds that inhibit an SGLT will require higher dosages. Less potent compounds include, for example, certain SGLT inhibitors such as O-glycosides.

Alternatively, fixed dosages may also be administered to felines that are independent of total body mass. In such embodiments, a therapeutically effective amount is a total daily dose of about 5, 10, 15, or 20 mg. In some embodiments, a therapeutically effective amount is a total daily dose of about 15 mg. A person of skill in the art will appreciate that less potent compounds that inhibit an SGLT will require higher dosages. Less potent compounds include, for example, certain SGLT inhibitors such as O-glycosides. These higher daily doses may include a therapeutically effective amount of about 50, 100, 150, or 200 mg, or a therapeutically effective amount of about 150 mg.

In some embodiments, the above-referenced amounts are suitable for treating felines with heart failure. In some embodiments, the above-referenced amounts are suitable for treating felines with CKD. In some embodiments, the above-referenced amounts are suitable for treating felines with hypertension.

In some embodiments, the therapeutically effective amount is one-time daily. In some embodiments, the therapeutically effective amount is two-times (twice) daily. In some embodiments, the therapeutically effective amount is three-times daily. In some embodiments, the therapeutically effective amount is four-times daily. Thus, dosages may be divided into equal or unequal portions administered at various time throughout the day.

In some embodiments, the therapeutically effective amount is an oral liquid dosage form. In some embodiments, the therapeutically effective amount is a solid dosage form.

Suitable amounts for other companion animals can be determined through clinical studies with these animals.

Combination therapy is also contemplated herein. In some embodiments, a companion animal with heart failure, hypertension, or chronic kidney disease receives an additional therapeutic agent. The additional therapeutic agent includes, but is not limited to, angiotensin-converting enzyme inhibitors (ACE inhibitors, such as enalapril, lisinopril and benazepril); angiotensin receptor blockers (such as valsartan and telmisartan); neprilysin inhibitors (such as sacubitril); diuretics, e.g. loop diuretics (such as furosemide and torsemide), thiazide diuretics (such as chlorthalidone and hydrochlorothiazide), and potassium-sparing diuretics (such as spironolactone); vasodilators (such as nitroglycerine, hydralazine and sodium nitroprusside); beta blockers (such as atenolol, metoprolol and propanolol); mixed β and α receptor antagonists (such as carvedilol); calcium channel blockers (such as amlodipine or diltiazem); mixed β-adrenergic receptor and potassium channel blockers (such as sotalol); and positive inotropes (such as pimobendan, digoxin, milrinone and dobutamine).

F. Pharmaceutical Compositions & Dosage Forms

The compounds and prodrugs that inhibit a sodium-dependent glucose transporter (SGLT) can be prepared in various compositions suitable for delivery to a companion animal. A composition suitable for administration to a companion animal typically comprises a compound or prodrug that inhibits an SGLT (or a pharmaceutically acceptable form thereof) and a pharmaceutically acceptable carrier.

Compounds or prodrugs that inhibit an SGLT (or a pharmaceutically acceptable form thereof) can be incorporated into a variety of formulations for therapeutic administration. More particularly, compounds or prodrugs that inhibit an SGLT can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of a compound of the present disclosure can be achieved in various ways, including oral, buccal, parenteral, intravenous, intradermal (e.g., subcutaneous, intramuscular), transdermal, etc., administration.

As recognized in the art, drugs for companion animals can be administered by a variety of means and in a variety of dosage forms. In some embodiments, oral dosage forms, such as oral liquid dosage forms, oral semi-solid dosage forms and oral solid dosage forms, including medicated foods and dosage forms are used. Because of the wide range in body masses of cats and dogs, oral liquid or semi-solid dosage forms that readily permit dosage adjustment for the mass of the animal are contemplated in the current disclosure.

Oral liquid dosage forms are often provided as a bottle plus a dispensing syringe or a calibrated dropper or if the medication is to be dispensed dropwise the bottle assembly may contain a narrow apical orifice and a flexible bottom portion allowing the contents to be expelled in drops by squeezing when the bottle is inverted.

Oral liquid dosage forms can be clear solutions, emulsions or suspensions, such as colloidal suspensions or coarse suspensions. They are often predominantly aqueous but may contain medically acceptable co-solvents such as ethanol, propylene glycol, glycerol or polyethylene glycols (also called PEGs or macrogols) of low molecular weight, such as PEG 200, PEG 300 or PEG 400. Oral liquid dosage forms may also be based on shelf-stable edible oils, such as canola, coconut, corn, cottonseed, olive, palm, peanut, safflower, sesame or sunflower oil, or mixtures thereof, or mixtures of glycerol or propylene glycol esterified with short or medium chain alkanoic acids with appropriately low melting points to exhibit liquefaction at room temperature. Non-metabolizable oils such as mineral oil may also be used but are less common bases for oral liquid formulations.

Oral liquid dosage forms containing ethanol are often referred to as elixirs and those containing sugars are often referred to as syrups. Oils and related nonaqueous solvents may be dispersed as emulsions in water, for example oil-in-water emulsions, or with the phases reversed, as water-in-oil emulsions. Oral liquid dosage forms may contain one or more solubilizers or surfactants such as ionic or nonionic detergents, as well as dispersants, thickening agents, emulsion or suspension stabilizers and agents to improve palatability (palatants, for example sodium acid pyrophosphate for feline dosage forms) or acceptability to the animal being treated, such as flavoring agents or sweeteners. Oral liquid dosage forms may also contain stability-promoting excipients such as buffers, anti-oxidants, and preservatives or anti-microbial agents. They may also be colored to provide a distinguishing characteristic to avoid inadvertent dosing with an inappropriate agent.

Pharmaceutically acceptable solubilizers and surfactants are well-known in the art and include ionic detergents such as ammonium or sodium lauryl (dodecyl) sulfate or sodium lauryl sulfosuccinate or sodium lauryl sulfoacetate or sodium lauroyl sarcosinate or cocobetaine. Other surfactants include nonionic detergents such as esterified sugars or dehydrated sugar mixtures such as sorbitan monoesters in the stearate, oleate, palmitate or laurate forms or oleate sesquiesters, that are frequently used to produce water-in-oil or oil-in-water emulsions, or bearing an additional pendant polyethoxyethanol side chain such as PEG-20 sorbitan isostearate or PEG-40 sorbitan diisostearate that are often used as emulsifiers, similar structures in which sorbitan is replaced by glycerol or propylene glycol, such as glyceryl stearate/PEG stearate, glyceryl stearate/PEG-100 stearate, lauroyl PEG-32 glycerides, PEG 6-32 stearate/glycol stearate, PEG-120 glyceryl stearate or various sugar ethers or esters such as PEG-120 methyl glucose dioleate, PEG-20 methyl glucose sesquistearate, alkanoyl glucosides or maltosides, fatty esters of polyethylene glycols such as PEG-5 oleate or stearate, or PEG-40 castor oil, or block copolymers of polypropylene glycol and polyethylene glycol, such as poloxamers. Nonionic detergents based octylphenoxy polyethoxyethanol, such as the octoxynols, bearing various lengths of the polyethylene glycol joined as an ether linkage to octylphenol are well-known in the art.

Various means to gauge the acceptability of an oral liquid dosage form to a dog or cat are well known in the art and include administration of the candidate dosage form to a diverse test population and determining the acceptability for example using various scoring systems based on behavioral responses to the test article, such as evasive or aversive responses, hypersalivation, foaming, spitting, vomiting or fractious behavior.

Bexagliflozin is well-tolerated by felines and canines and does not seem to have an objectionable flavor for animals of either species. Taste masking to prevent aversive responses to bexagliflozin itself is rarely necessary but may be helpful to promote acceptance of excipients in the formulation. Felines lack sweet taste receptors and sweeteners are less important for feline oral liquid dosage forms. Bexagliflozin has a solubility in water at 25° C. of approximately 0.5 mg mL⁻¹, but is more soluble in ethanol, propylene glycol and PEGs.

### IV. Examples

The following examples are offered for illustrative purposes, and are not intended to limit the disclosure in any manner. Those of skill in the art will readily recognize a variety of noncritical parameters which can be changed or modified to yield essentially the same results.

### Example 1: Bexagliflozin Effects on Thiazolidinedione-Mediated Fluid Retention in Rats

To evaluate the ability of bexagliflozin to reduce the fluid retention associated with heart failure, the PPAR-γ-mediated fluid retention model was selected. It has been recognized in human clinical practice that an undesirable side effect of PPAR-γ agonists of the thiazolidinedione class is fluid retention and edema, which limits their use in patients with congestive heart failure. The mechanism of action of the thiazolidinediones is believed to be through stimulation of expression of the epithelial sodium channel (ENaC) in the kidney, resulting in an increased uptake of sodium ions from the distal tubule. Sodium retention and the associated expansion of extracellular fluid volume are characteristic features of congestive heart failure in multiple species. ENaC blockers such as amiloride have been shown to prevent PPAR-γ-mediated fluid retention in mice (Guan et al., 2005, Nat Med 11:861; doi: 10.1038/nm1278) and rats (Chen et al., 2005, J Pharmacol Exp Ther 2312:718; doi: 10.1124/jpet.104.074088).

A study was undertaken to assess the ability of bexagliflozin delivered in drinking water for 7 days to prevent PPAR-γ agonist-mediated plasma expansion in Sprague-Dawley rats. The sodium channel blocker amiloride was delivered in drinking water to a third group of rats as a comparator. Plasma expansion was measured by hematocrit.

On day 1, rats were weighed and received an admixed diet (ad lib) containing 0.03% pioglitazone in place of standard chow. Food weight was recorded to measure consumption over the course of the experiment. Rats were also given drinking water containing bexagliflozin or amiloride at the appropriate concentrations. Full bottles were pre-weighed and the weights recorded to allow consumption to be measured over the course of the experiment.

On days 1 and 7, rats were warmed using a heating pad and lightly anesthetized using isoflurane. Using a sterile scalpel blade, a small tail nick was made on the distal one third of the tail. Blood was collected in a heparinized microcapillary tube sealed with putty at one end and stored on ice until centrifugation (5000 rpm, 5 min). Plasma volume was determined using a microhematocrit capillary tube reader card.

On day 7, rats, food and water bottles were again weighed and recorded and hematocrits were determined using blood collected by a tail nick.

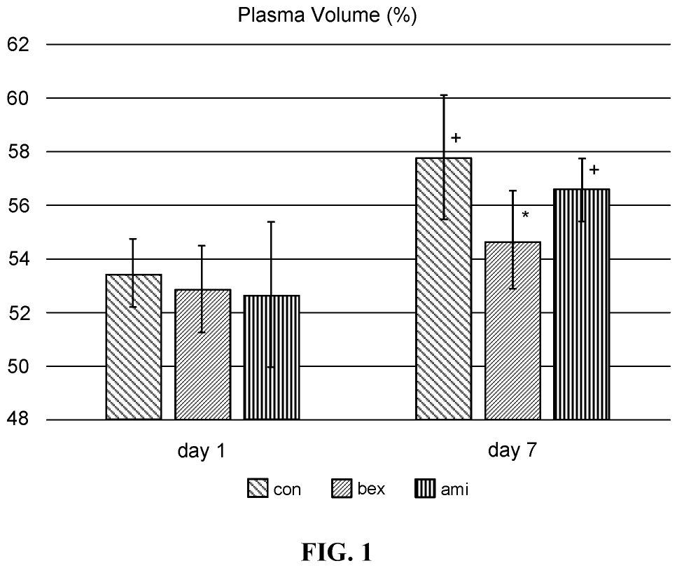

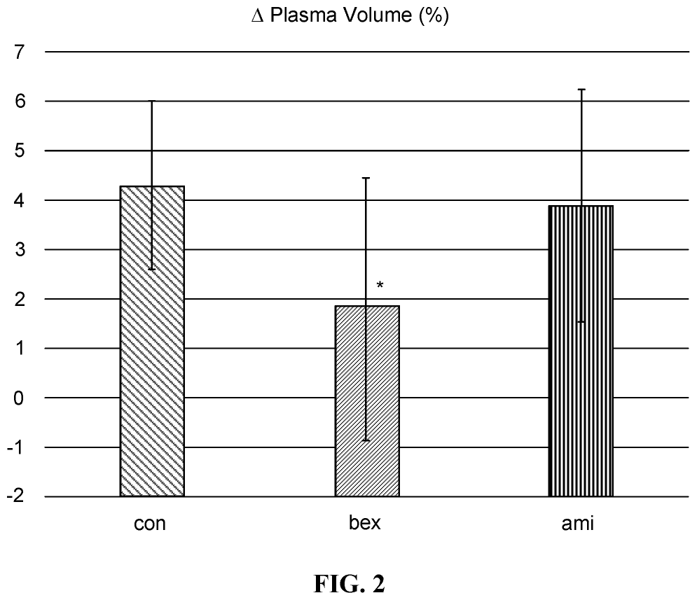

Significant main effects of time (F_{(1,27)}=64.9; p<0.0001) and treatment (F_{(2,27)}=3.73; p=0.037), as well as a significant interaction (F_{(2,27)}=3.51; p=0.044) were revealed by 2-way ANOVA. Day 1 mean plasma volumes were similar (approximately 53% of blood volume) among the treatment groups (F_{(2,27)}=0.45; p=0.64). However, after consuming a pioglitazone-containing diet for 7 days, plasma volumes were significantly different (F_{(2,27)}=7.32; p=0.003), with bexagliflozin-treated rats displaying significantly less plasma expansion than vehicle treated rats (p<0.01) (FIG. 1 and Table 1). Although day 7 plasma volumes were significantly increased compared to day 1 plasma volumes in rats receiving vehicle or amiloride (p<0.001), plasma expansion in bexagliflozin treated rats did not reach significance (FIG. 1 and Table 1). Analysis of the change in plasma volume resulted in similar results (F_{(2,27)}=3.51; p<0.044) (FIG. 2 and Table 1). Pair-wise comparisons of the change in volume were also similar to those from the day 7 analyses, as bexagliflozin-treated rats displayed significantly less plasma expansion than vehicle-treated rats (p<0.05).

Significant main effects of time (F_{(1,27)}=303.8; p<0.0001) and treatment (F_{(2,27)}=18.6; p<0.0001), as well as a significant interaction (F_{(2,27)}=18.6; p<0.0001) were revealed by the 2-way ANOVA of the body mass gain. Animals were assigned to treatment groups stratified by body mass, and body mass gains were expressed as gains from day 1 values. Mass gain was observed over the course of the experiment, with a significant difference among day 7 group means expressed as percent mass gain (F_{(2,27)}=18.6; p<0.0001; Table 2). All rats gained significant mass over the 7 days of treatment (p<0.0001). However, those receiving bexagliflozin gained significantly less mass than those receiving vehicle; 4.8% gain compared to 12.3% gain (p<0.01). Analysis of the change in mass resulted in similar test statistic results (F_{(2,27)}=18.6; p<0.0001; Table 2). Pair-wise comparisons were also similar to those from the day 7 analyses, as bexagliflozin-treated rats displayed significantly less mass gain than untreated rats (p≤0.01). The increase in body mass in the untreated rats is explicable as a combination of the normal gain of a growing rat and the extracellular fluid volume expansion promoted by pioglitazone. The lower mass exhibited by bexagliflozin-treated rats is consistent with the effects observed on plasma volume, although effects on weight gain due to caloric wasting as a result of glucosuria may also have been present.

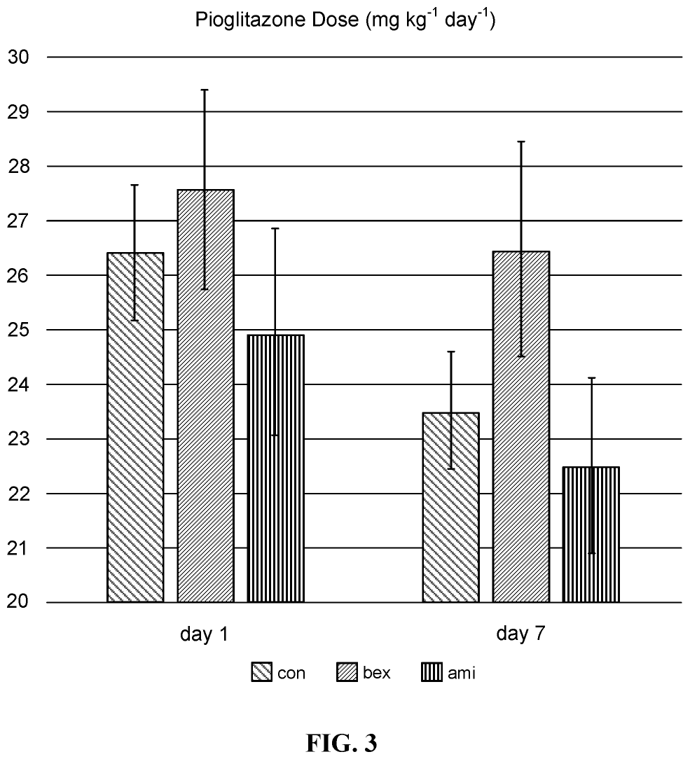

However, daily food consumption was similar between treatment groups with slightly lower consumption observed in the amiloride treated rats and slightly higher consumption observed in the bexagliflozin treated rats, resulting in a significant overall test statistic (F_{(2,27)}=3.94; p=0.032). Neither treatment group was found to consume a significantly different amount of food than vehicle treated rats (Table 3). As a result, animals received similar levels of pioglitazone exposure (FIG. 3), with the bexagliflozin cohort receiving the highest dose. Thus, a reduction in plasma volume expansion was detected despite a higher dose of pioglitazone.

Daily water consumption was significantly different between treatment groups over the 7 days of the experiment (F_{(2,27)}=53.8; p<0.0001). This was attributed to the previously recognized tendency of bexagliflozin to increase water consumption to compensate for diuresis. The intergroup difference was significant between the bexagliflozin and control cohorts (p≤0.01; Table 4). Daily doses of bexagliflozin and amiloride were about 50% higher than targeted, resulting in approximately 4.5 mg kg⁻¹ day⁻¹ bexagliflozin and 1.5 mg kg⁻¹ day⁻¹ amiloride (Table 4).

The results of this experiment indicate that bexagliflozin substantially attenuates pioglitazone-mediated plasma expansion. The intended positive control, amiloride, was markedly less effective than bexagliflozin and did not produce the anticipated effect. These data support the utility of bexagliflozin for the management of heart failure in dogs and cats.

TABLE 1
Effects of Bexagliflozin or Amiloride on Plasma Volume

| Group | Control | Bexagliflozin | Amiloride |
|---|---|---|---|
| Day 1 | 53.5 ± 0.4 | 52.9 ± 0.5 | 52.7 ± 0.9 |
| Day 7 | 57.8 ± 0.7 ^{††} | 54.7 ± 0.6 ** | 56.6 ± 0.4 ^{††} |
| Δ | 4.3 ± 0.5 | 1.8 ± 0.8 * | 3.9 ± 0.7 |

Values represent group means ± SEM;
* p ≤ 0.05, ** p ≤ 0.01 (compared to control);
^{††} p ≤ 0.01 (compared to day 1)

TABLE 2
Effects of Bexagliflozin or Amiloride on Body Mass

| Group | | Control | Bexagliflozin | Amiloride |
|---|---|---|---|---|
| Body | Day 1 | 372.8 ± 3.5 | 371.7 ± 5.0 | 370.0 ± 6.0 |
| Weight | Day 7 | 418.7 ± 4.6 ^{††} | 389.2 ± 5.7 ** ^{††} | 410.0 ± 7.7 ^{††} |
| (g) | Δ | 45.9 ± 2.6 | 17.5 ± 4.7 ** | 40.0 ± 2.8 |
| Body | Day 1 | 100 ± 0 | 100 ± 0 | 100 ± 0 |
| Weight | Day 7 | 112.3 ± 0.7 ^{††} | 104.8 ± 1.3 ** ^{††} | 110.8 ± 0.7 ^{††} |
| (%) | Δ | 12.3 ± 0.7 | 4.8 ± 1.3 ** | 10.8 ± 0.7 |

Values represent group means ± SEM;
* p ≤ 0.05, ** p ≤ 0.01 (vs control);
^{††} p ≤ 0.01 (vs day 1)

TABLE 3
Effects of Bexagliflozin or Amiloride on Daily Food Consumption

| Group | Control | Bexagliflozin | Amiloride |
|---|---|---|---|
| Food Consumption | 32.8 ± 0.5 | 34.4 ± 1.0 | 30.9 ± 1.1 |
| (g per 24 h) | | | |
| Day 1 Dose | 26.4 ± 0.4 | 27.7 ± 0.6 | 25.0 ± 0.6 |
| Day 7 Dose | 23.5 ± 0.3 | 26.5 ± 0.6 | 22.5 ± 0.5 |

Values represent group means ± standard error of measurement

TABLE 4
Effects of Bexagliflozin or Amiloride on Water Consumption

| Group | Control | Bexagliflozin | Amiloride |
|---|---|---|---|
| Water Consumption | 49.0 ± 1.9 | 80.2 ± 2.8 ** | 46.9 ± 2.8 |
| (24 h) | | | |
| Day 1 Dose | | 4.5 ± 0.13 | 1.5 ± 0.07 |
| Day 7 Dose | | 4.3 ± 0.15 | 1.3 ± 0.07 |

Values represent group means ± SEM;
** p ≤ 0.01 (vs control)

### Example 2: Bexagliflozin Effects in Dogs

The effects of bexagliflozin in dogs were assessed in a study evaluating the pharmacokinetics and pharmacodynamics of the compound delivered by oral gavage. Groups of four purpose-bred beagles per cohort were administered ascending doses of bexagliflozin in the form of a proline co-crystal consisting of two moles of L-proline for every mole of bexagliflozin. The co-crystal was dissolved in 10% PEG400 and administered by oral gavage to dogs that had been fasted overnight. Control dogs were administered 10% PEG400. One hour after dosing, glucose solution (2 g kg⁻¹, 5 ml kg⁻¹) was administered by gavage. Serum glucose concentrations were quantitated by an automated clinical chemistry analyzer for samples collected before dosing, before glucose challenge, and 15, 30, 60, and 120 min after glucose challenge. Urine was collected from 0 to 4 h, 4 to 8 h and 8 to 24 h after dosing for electrolyte analyses (Na⁺, K⁺, Cl⁻ and Ca²⁺) and glucose measurement. Whole blood samples of approximately 500 μL were collected from the cephalic vein 0, 0.25, 0.5, 1, 2, 4, 8, 12 and 24 h after dosing for pharmacokinetic analysis. Doses ranged in strength from 0.02 to 2.0 mg kg⁻¹ bexagliflozin.

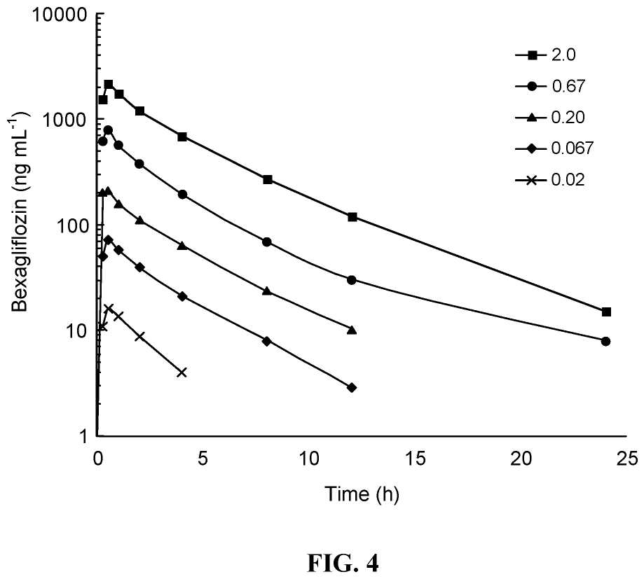

Bexagliflozin was rapidly absorbed with a Tₘₐₓ of approximately 0.5 h. Cₘₐₓ and AUC were roughly dose proportional over the entire dosing range and the elimination half-life ranged from 1.67 to 5.23 h (Table 5 and FIG. 4). Only modest changes in serum glucose concentration were seen around the time of administration of glucose by gavage (Table 6), and presumably reflect bexagliflozin blockade of enteric uptake of glucose, assumed to be an effect on SGLT1.

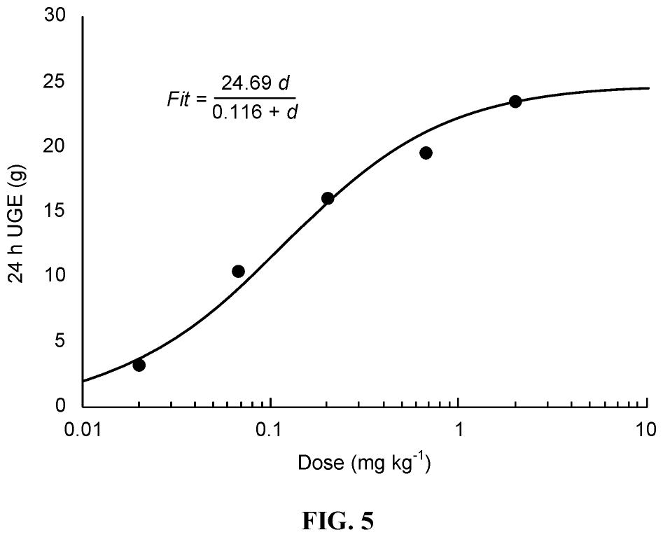

A good correlation was seen between glucosuria and exposure to bexagliflozin and the urinary glucose excretion was fit well to the dose by a simple logistic model with an ED₅₀ of 0.116 mg kg⁻¹ and an Eₘₐₓ of 24.69 g of glucose in 24 h (FIG. 5). Urine output increased in the treatment group, consistent with an osmotic diuresis or with a role for the inhibited transporters in water reuptake in the proximal tubule. Trends toward increased urinary electrolyte excretion were seen in the treatment group. Table 7 shows that the increase in urine volume and urine Na⁺ content was statistically significant for dogs in the highest dose group compared to dogs in the vehicle group. There was no significant change in food consumption in the treatment group in comparison with the control. A trend toward higher water consumption was observed in the treatment group but did not reach statistical significance.

Although many factors influence the variation in pharmacological activity of compounds administered to experimental animals, the practical import of the identification of the potent effect of bexagliflozin on dogs can be understood in relation to the previously characterized activity of the compound in cats. Cats administered bexagliflozin tablets of varying strengths have been found to respond with a urinary glucose excretion that is well-described by a simple logistic model with an ED₅₀ of approximately 0.5 mg kg⁻¹, which makes bexagliflozin approximately 4 times more potent by ED₅₀ in dogs than in cats. The average mass of the dogs in the study above was 5.84 kg, leading to a maximum urinary glucose excretion of 4.23 g glucose per kg of body mass. Comparable studies of healthy cats have produced an Eₘₐₓ of 2.21 g of glucose per kg of body mass, indicating that bexagliflozin has a more profound effect on canine glucose disposition than on feline disposition. Inasmuch as the effects on renal physiology are mediated at the level of the nephron, it is clear that canine and feline responses to bexagliflozin are highly similar and effects found in cats are likely to be replicated in dogs. In particular, it can be anticipated that renal disease in dogs can be managed with bexagliflozin in the same way as for cats.

TABLE 5
Bexagliflozin Pharmacokinetic Parameters in Dogs

| Dose | Tₘₐₓ | Cₘₐₓ | AUC₀₋₂₄ | AUC_{0-∞} | t_{1/2} | CL/F | Vz/F |
|---|---|---|---|---|---|---|---|
| mg kg⁻¹ | h | ng mL⁻¹ | ng h mL⁻¹ | ng h mL⁻¹ | h | mL h⁻¹ kg⁻¹ | mL kg⁻¹ |
| 0.02 | 0.50 | 16.3 ± 2.7 | 35.6 ± 5.2 | 45.3 ± 7.9 | 1.67 ± 0.26 | 456 ± 95 | 1076 ± 128 |
| 0.067 | 0.50 | 72.9 ± 11.0 | 240 ± 55 | 257 ± 62 | 2.81 ± 0.61 | 272 ± 64 | 1062 ± 67 |
| 0.20 | 0.25 | 233 ± 26 | 725 ± 80 | 769 ± 95 | 2.95 ± 0.28 | 265 ± 35 | 1117 ± 80 |
| 0.67 | 0.50 | 801 ± 46 | 2603 ± 104 | 2666 ± 131 | 5.23 ± 1.23 | 252 ± 12 | 1886 ± 398 |
| 2.0 | 0.50 | 2199 ± 539 | 8605 ± 1523 | 8696 ± 1472 | 3.89 ± 0.88 | 237 ± 46 | 1370 ± 601 |

Values represent mean ± SD (n = 4) except for Tₘₐₓ (median). Parameters were estimated by noncompartmental methods using commercially available software (WinNonlin).

TABLE 6
Serum Glucose Concentration (mg dL⁻¹) After a Single Oral Dose
of Bexagliflozin in Dogs Challenged with Glucose 1 h Post-Dose

| Dose mg | | | | | | |
|---|---|---|---|---|---|---|
| kg⁻¹ | −60 min | 0 min | 15 min | 30 min | 60 min | 120 min |
| 0 | 104.72 ± 8.71 | 104.40 ± 9.78 | 136.89 ± 20.45 | 128.07 ± 19.46 | 124.11 ± 24.21 | 90.41 ± 1.84 |
| 0.02 | 100.89 ± 3.49 | 95.94 ± 8.37 | 122.13 ± 17.54 | 97.43 ± 6.87 | 93.29 ± 19.87 | 78.03 ± 10.98 |
| 0.067 | 98.64 ± 7.63 | 91.58 ± 6.49* | 107.91 ± 12.08* | 104.31 ± 26.84 | 104.27 ± 17.85 | 79.65 ± 11.69 |
| 0.20 | 97.02 ± 7.61 | 84.65 ± 7.98** | 96.71 ± 7.21** | 105.48 ± 18.67 | 101.75 ± 14.83 | 76.10 ± 7.91 |
| 0.67 | 97.70 ± 5.27 | 87.08 ± 6.03** | 109.94 ± 17.55* | 109.04 ± 26.58 | 106.79 ± 11.98 | 76.95 ± 11.53 |
| 2.0 | 92.21 ± 7.11 | 85.10 ± 4.74** | 104.49 ± 9.60** | 112.86 ± 9.82 | 108.81 ± 6.77 | 71.06 ± 5.49 |

*p < 0.05; **p < 0.01 compared to vehicle cohort. Values represent the mean ± SD (n = 4).

TABLE 7
Bexagliflozin Effects on Electrolytes in 24 h Urine Collections in Dogs

| Dose | | | | |
|---|---|---|---|---|
| mg kg⁻¹ | Volume mL | Na⁺ mg | K⁺ mg | Cl⁻ mg |
| 0.0 | 159.4 ± 68.7 | 222.1 ± 74.5 | 516.6 ± 106.5 | 431.1 ± 106.1 |
| 0.02 | 118.0 ± 30.8 | 178.7 ± 16.7 | 426.2 ± 62.1 | 307.4 ± 87.7 |
| 0.067 | 153.1 ± 44.5 | 220.9 ± 53.9 | 476.8 ± 96.5 | 403.4 ± 111.4 |
| 0.20 | 214.1 ± 53.5 | 275.7 ± 69.5 | 558.8 ± 118.2 | 526.5 ± 127.8 |
| 0.67 | 225.0 ± 66.9 | 300.2 ± 66.4 | 587.7 ± 113.5 | 573.9 ± 166.5 |
| 2.0 | 247.7 ± 35.2* | 313.1 ± 35.1* | 599.7 ± 93.5 | 595.8 ± 80.6 |

*p < 0.05 for comparison to vehicle cohort. Values represent mean ± SD (n = 4).

As mentioned above, loss of body and muscle mass is a common comorbidity of canine heart failure and cachexia is a manifestation of end-stage disease. Bexagliflozin causes a profound loss of glucose in dogs and as such would be expected to exacerbate the clinical condition of dogs with heart failure. Hence, it is counterintuitive that bexagliflozin will reduce the progression of the clinical condition of dogs in this state, and especially unexpected that bexagliflozin will promote an improvement in musculature in some animals, despite the wasting influence typically evidenced in its most severe form as a heart failure-related cachexia.

### Example 3: Bexagliflozin Effects on Measures of Renal Health in Cats with Diabetes

In studies exploring the utility of bexagliflozin for the management of feline diabetes, animals with diabetes were presented for veterinary care and were, if eligible according to the enrollment criteria, with their owners' consent entered into one or more studies investigating the effects of bexagliflozin on glycemic control and clinical signs of hyperglycemia. Venous blood specimens were collected at regular intervals and analyzed for standard parameters of interest for routine clinical care, including creatinine, BUN and SDMA. Owners were advised to provide a diet low in carbohydrates as an element of the disease management plan. An important consequence of the shift to a low carbohydrate diet is the increase in dietary protein (contributing to BUN) and specifically dietary muscle-derived protein (contributing to creatinine).

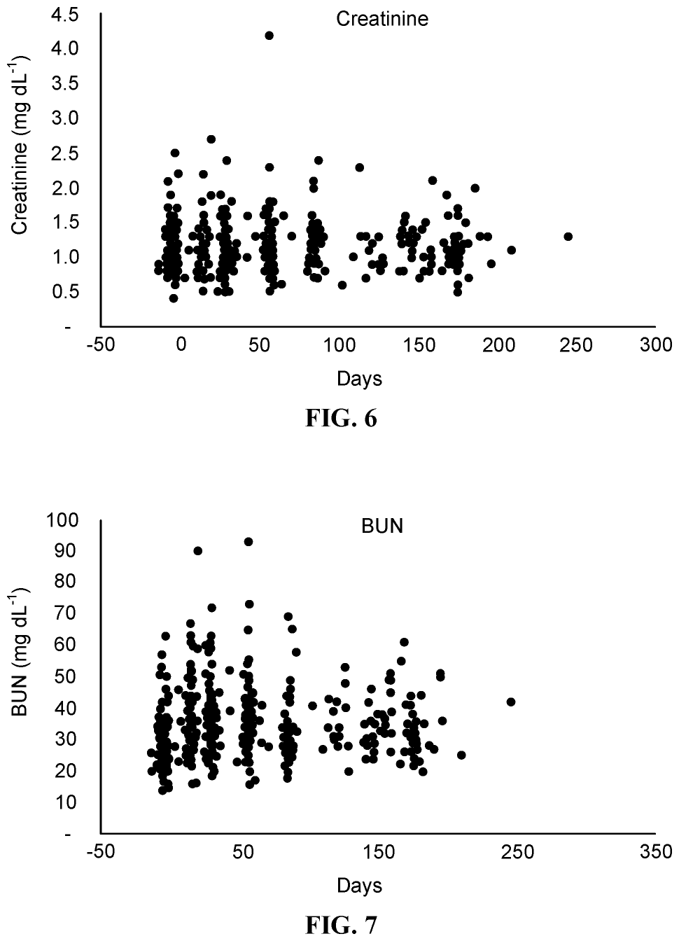

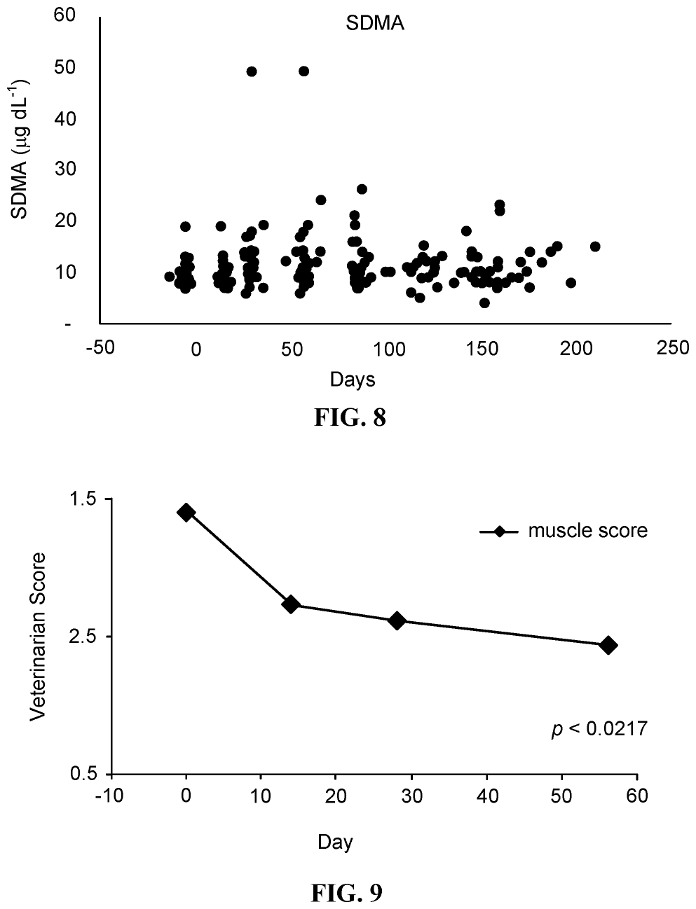

FIG. 6, FIG. 7 and FIG. 8 show the measured concentrations of creatinine, BUN and SDMA in cats as a function of time in the study. The initial measurement precedes the administration of bexagliflozin and the change in diet. Considerable variation can be seen in the individual values.

To identify treatment-related trends, the data for each of the three analytes were analyzed by a mixed model repeated measures (MMRM) analysis of covariance, with cat as a categorical (class) effect and day of measurement and initial value at the baseline visit as continuous covariates. Because the diets of many of the animals were changed at the baseline visit, and the effects of diet on creatinine and BUN are generally thought to be prompt, the analysis considered only changes in analyte values over the non-baseline visit days.

The model-adjusted estimate for the population mean creatinine concentration was 1.367 mg dL⁻¹, with an estimated coefficient for day of measurement of 0.000928. Day of measurement was a highly significant effect, p<0.0001.

The model-adjusted estimate for the population mean BUN concentration was 33.82 mg dL⁻¹, with an estimated coefficient for day of measurement of −0.01346 (i.e. the slope of the BUN concentration with time was negative, signifying decreasing BUN). Day of measurement was a significant effect, p=0.0172.

The model-adjusted estimate for the population mean SDMA concentration was 19.10 μg dL⁻¹, with an estimated coefficient for day of measurement of −0.0021 (signifying decreasing SDMA). Day of measurement was not a significant effect, p=0.5338.

As discussed above, creatinine, BUN and SDMA are related measures of renal health for which increasing concentration signifies a dwindling renal function. It is therefore unexpected that the trends diverge, with creatinine increasing, and BUN and SDMA decreasing.

The key to reconciling this apparent discrepancy lies in other factors that influence creatinine concentration that are consequences of the effective management of diabetes with bexagliflozin.

Cats presented by owners to veterinarians for diabetes are often in a state of advanced disease and occasionally moribund. Weight loss despite an apparently ravenous appetite is frequently observed, and is one of the well-recognized symptoms of the disease, which is also marked by polydipsia (excessive drinking), polyphagia (excessive food consumption) and polyuria (excessive urination). The beneficial effects of bexagliflozin for promoting weight gain and reducing the clinical signs of hyperglycemia have been disclosed in U.S. Patent Application No. 62/818,589.

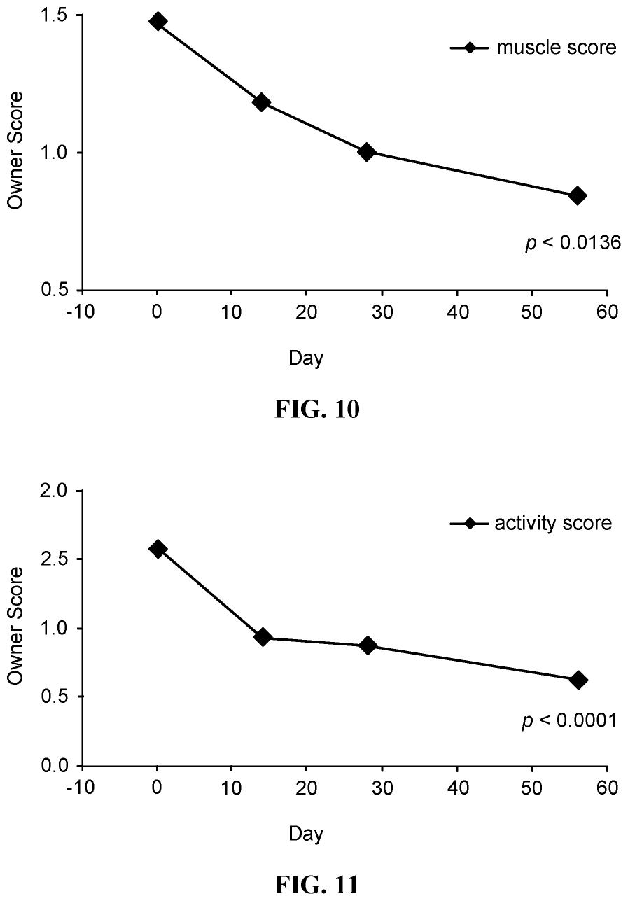

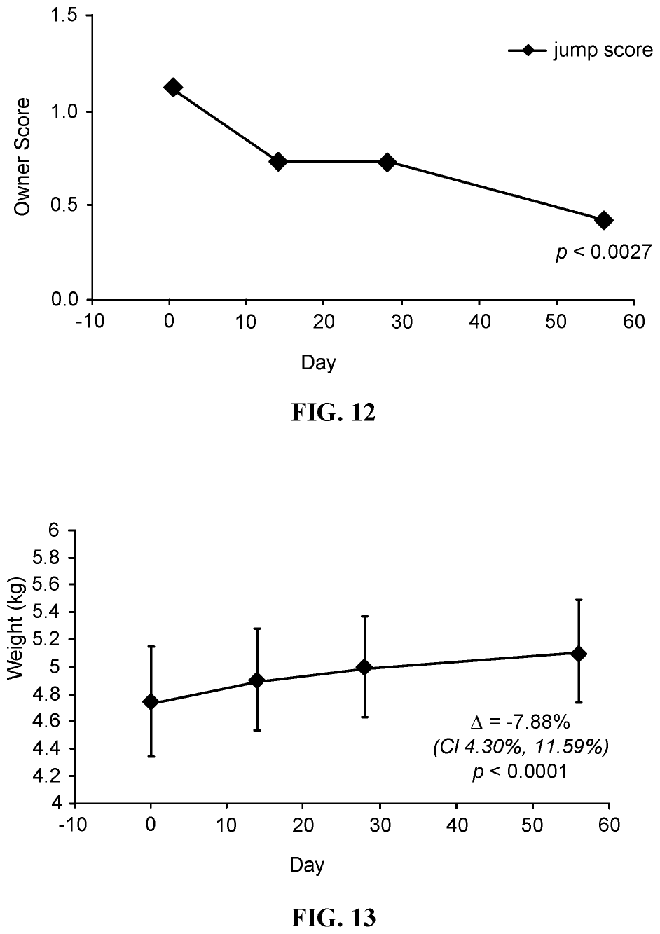

Assessments by both owners and veterinarians of correlative measures of cat vitality were also collected over the course of various studies. Some of the relevant measures for this analysis are the subjective impressions of cat musculature, solicited from both owners and veterinarians, and owner views of cat overall activity as well as leaping ability. The rating instrument was a 4-point scale with 0=excellent, 1=good, 2=fair and 3=poor. FIG. 9 and FIG. 10 show that both veterinarians and owners, respectively considered that the musculature of study cats had improved, and the effect was statistically significant (p-values are shown in a figure inset). FIG. 11 and FIG. 12 show that owners considered that their cats were more active and had better leaping ability. FIG. 13 shows that the mean cat weight increased, and that the effect was highly significant (the confidence intervals show the population variation, whereas the effect for individual cats relative to their weights at study entry was highly significant).

Together, these data provide a picture of increasing vitality among the enrolled cats that is accompanied by an increase in weight and musculature. Hence, in this context it can be appreciated that increasing musculature and activity can account for an increase in creatinine even when broader and more sensitive indicators of renal dysfunction point to improvements in health. Even though creatinine is often the reference measurement for renal assessments, the more sensitive and putatively more specific SDMA assay did not corroborate the creatinine predictions.

### Example 4: Bexagliflozin Effects on Measures of Cardiac Health in Dogs and Cats with Heart Failure

The natriuretic and diuretic actions of bexagliflozin will benefit dogs and cats with heart failure by providing relief from the systemic adverse consequences of hypervolemia and fluid retention, as demonstrated by the prevention of pioglitazone-mediated fluid retention in rats. In addition, bexagliflozin can improve the overall health and vitality of the animal, attenuating or even reversing the deleterious consequences of chronic heart overload on skeletal muscle mass and fitness.

### Example 5: Bexagliflozin Treatment of Dogs with Heart Failure

Bexagliflozin can be administered to dogs with heart failure as an oral solution, such as a flavored oral solution, or as a combination of tablets and/or half-tablets to allow delivery of a dose adjusted for body mass, such 50 μg kg⁻¹, 100 μg kg⁻¹, 200 μg kg⁻¹, 400 μg kg⁻¹, 800 μg kg⁻¹, 1600 μg kg⁻¹ or 3200 μg kg⁻¹. These doses will produce 30%, 46%, 63%, 77%, 87%, 93%, 96% or 98% of the maximum pharmacodynamic effect based on the results observed in beagle dogs. Doses may be adjusted as necessary to accommodate the needs of the dog, for example reduced if deleterious effects such as diarrhea or vomiting are observed, or increased if the desired pharmacological effect has not been reached. Dosage may be divided, for example into two equal portions to be administered approximately 12 h apart. A dose of approximately 1 mg kg⁻¹ delivered once daily will produce approximately 90% of the maximum pharmacological effect and is likely to represent a frequently prescribed dosage level for dogs with heart failure. Dogs exposed to this amount of bexagliflozin do not show signs of enteric inhibition of SGLT1, for example as manifested by diarrhea or loose stools, although they may initially consume more water and tend to eat more than usual to compensate for the loss of fluid and calories as a result of the glucosuria. Supplementation of the diet may be necessary to promote the maximum vitality of the dog. Bexagliflozin-treated dogs will show increased activity, for example, more spontaneous activity and more willingness to engage with their owners in play. They will need to urinate more and should be provided with an ample supply of clean water to replenish their extracellular volume once the excess volume has been shed. Dogs with heart failure managed by bexagliflozin will experience fewer episodes of volume expansion requiring hospitalization or intensive intervention to alleviate pulmonary edema and its clinical signs. Dogs with heart failure managed by bexagliflozin will, compared to dogs not so managed, experience a longer time to one or more of the following: (i) cardiac death, (ii) euthanasia for heart failure or (iii) evidence of treatment failure such as persistent dyspnea, progressive ascites, severe cardiac cachexia, or severe exercise intolerance despite receiving or failing to tolerate a diuretic dosage of furosemide (12 mg kg⁻¹ day⁻¹) and spironolactone (6 mg kg⁻¹ day⁻¹). Owner mean ratings of dog musculature will, on average, improve as a result of the salutary effects of bexagliflozin, even though bexagliflozin promotes caloric wasting and would be anticipated to result in an exacerbation of cardiac cachexia and its prodromes. Bexagliflozin will be useful for the management of the signs of mild, moderate, or severe (modified NYHA Class II, III, or IV) congestive heart failure in dogs due to atrioventricular valvular insufficiency or dilated cardiomyopathy. Bexagliflozin can be dosed with other medications for the management of heart failure, such as pimobendan, furosemide, RAS blocking agents and spironolactone.

### Example 6: Bexagliflozin Treatment of Cats with Heart Failure

Bexagliflozin can be administered to cats with heart failure as an oral solid dosage form, such as a tablet, or as an oral solution, such as a flavored oral solution, to allow delivery of a dose adjusted for body mass, such 200 μg kg⁻¹, 400 μg kg⁻¹, 800 μg kg⁻¹, 1600 μg kg⁻¹, 3200 μg kg⁻¹, 6400 μg kg⁻¹, 12800 μg kg⁻¹ or 25600 μg kg⁻¹. These doses will produce 28%, 44%, 61%, 76%, 86%, 93%, 96% or 98% of the maximum pharmacodynamic effect based on the results observed in purpose-bred cats. Doses may be adjusted as necessary to accommodate the needs of the cat, for example, reduced if deleterious effects such as diarrhea or vomiting are observed, or increased if the desired pharmacological effect has not been reached. A dose of 2.5 mg kg⁻¹ to 5 mg kg⁻¹ delivered once daily will produce approximately 83% to 91% of the maximum pharmacological effect and is likely to represent a frequently prescribed dosage range for cats with heart failure. Dosage may be divided, for example, into two equal portions to be administered approximately 12 h apart. Alternatively, a single dosage strength of 15 mg, for example, a tablet containing 15 mg of bexagliflozin, can be administered to a cat without adjustment for body mass. The fixed dosage can also be divided into equal portions for b.i.d. dosing if desired. Cats exposed to this amount of bexagliflozin can occasionally show signs of enteric inhibition of SGLT1, for example, as manifested by diarrhea or loose stools, for which a diet containing a higher proportion of metabolizable energy in the form of protein or fat may be advised. Diarrhea and loose stools are generally dependent on carbohydrate in the diet and low-carbohydrate diets are preferred if enteric side effects are observed. Supplementation of the diet may be necessary to promote the maximum vitality of the cat. Bexagliflozin-treated cats often show increased activity, for example, more spontaneous activity and display enhanced abilities to jump as reported by their owners. They will need to urinate more and should be provided with an ample supply of clean water to replenish their extracellular volume once the excess volume has been shed. Cats with heart failure managed by bexagliflozin will experience fewer episodes of volume expansion requiring hospitalization or intensive intervention to alleviate pulmonary edema and its clinical signs. Cats with heart failure managed by bexagliflozin will, compared to cats not so managed, experience a longer time to one or more of the following: (i) death (including euthanasia) or (ii) withdrawal of the cat from treatment because of worsening of clinical condition related to heart disease, such as a persistent and unacceptably high heart rate, a need for repeated thoracocentesis to alleviate pulmonary edema, or a ventricular arrhythmia requiring treatment. Owner mean ratings of cat musculature will, on average, improve as a result of the salutary effects of bexagliflozin, even though bexagliflozin promotes caloric wasting and would be anticipated to result in an exacerbation of cardiac cachexia and its prodromes. Bexagliflozin will be useful for the management of the signs of mild, moderate, or severe (modified NYHA Class II, 111, or IV) congestive heart failure in cats due to atrioventricular valvular insufficiency or dilated cardiomyopathy. Bexagliflozin can be dosed with other medications for the management of heart failure, such as pimobendan, furosemide, RAS blocking agents and spironolactone.

### Example 7: Bexagliflozin Treatment of Dogs with Chronic Kidney Disease

Bexagliflozin can be administered to dogs with chronic kidney disease as described above for the treatment of heart failure. A dose of 1 mg kg⁻¹ will produce approximately 90% of the maximum pharmacological effect and is likely to represent a frequently prescribed dosage level for dogs with CKD. Dogs administered bexagliflozin will tolerate carbohydrate in the diet well, but reduction in the amount of carbohydrate may be necessary if persistent diarrhea or loose stools are observed. Dogs on a renal diet may need supplementation in the number of daily calories provided to accommodate the effects of the renal glucosuria. A preferred supplementation is by a mixture of edible fats and carbohydrates, as a reduced protein content is preferred. Dogs on a renal diet will still benefit from the myoprotective effects of bexagliflozin, although these may be manifest as a reduced rate of muscle loss instead of overt muscle gain. Dogs with CKD, especially dogs with proteinuria, will experience a treatment-related benefit defined as a composite of the occurrence of death (including euthanasia) or the need for administration of parenteral fluids related to renal failure. Dogs with CKD will experience a slowing in the rate of progression of proteinuria, measured by the urinary protein to creatinine ratio. Some dogs with signs of muscle loss or cachexia will gain weight as a result of the actions of bexagliflozin on renal health. Bexagliflozin will, in general, retard the advancement of CKD in dogs but is not expected to meaningfully affect the health or vitality of dogs with stage D CKD, as dogs with this degree of renal impairment may have very little renal filtration capacity and hence there is little opportunity for the palliative consequences of bexagliflozin on renal transporters to be exerted.

### Example 8: Bexagliflozin Treatment of Cats with Chronic Kidney Disease

Bexagliflozin can be administered to cats with chronic kidney disease as described above for the treatment of heart failure. A dose of 2.5 mg kg⁻¹ to 5 mg kg⁻¹ will produce approximately 83% to 91% of the maximum pharmacological effect and is likely to represent a frequently prescribed dosage range for cats with CKD. A single dosage strength of 15 mg, for example a tablet containing 15 mg of bexagliflozin, can be administered to a cat without adjustment for body mass. The fixed dosage can also be divided into equal portions for bid dosing if desired. Cats administered bexagliflozin may experience enteric side effects and a reduction in the amount of carbohydrate may be necessary if persistent diarrhea or loose stools are observed. Cats on a renal diet may need supplementation in the number of daily calories provided to accommodate the effects of the renal glucosuria. A preferred supplementation is by a mixture of edible fats and carbohydrates, as a reduced protein content is desirable. Cats on a renal diet will still benefit from the myoprotective effects of bexagliflozin, although these may be manifest as a reduced rate of muscle loss instead of overt muscle gain. Cats with CKD, especially cats with proteinuria, will experience a treatment-related benefit defined as a composite of the occurrence of death (including euthanasia) or the need for administration of parenteral fluids related to renal failure. Cats with CKD will experience a slowing in the rate of progression of proteinuria, measured by the urinary protein to creatinine ratio. Bexagliflozin will retard the advancement of CKD in cats but is not expected to meaningfully affect the health or vitality of cats with stage D CKD, as cats with this degree of renal impairment may have very little renal filtration capacity and hence there is little opportunity for the palliative consequences of bexagliflozin on renal transporters to be exerted.

### Example 9: Bexagliflozin Treatment of Dogs with Hypertension

Bexagliflozin produces a reduction in hypertension by a combination of osmotic diuresis and effects on tubuloglomerular feedback exerted at the level of the macula densa. The hypotensive effect of bexagliflozin is mild, but the overall effects are beneficial for the health of the dog. Dogs with hypertension managed by bexagliflozin will typically lose a small amount of weight which will be beneficial if they are overweight or obese—as is often the case for hypertensive dogs—but will otherwise be healthy. Dogs with hypertension managed with bexagliflozin will need to have adequate water provided to accommodate their increased urine output. A supplementation of the diet may be necessary. If diarrhea or loose stools are observed, the dose can be reduced or the diet changed to provide a decreased fraction of metabolizable energy in the form of carbohydrates. Dogs will tolerate bexagliflozin well and owners will express satisfaction with the health of their dogs compared to owners of dogs whose disease is not managed by bexagliflozin.

### Example 10: Bexagliflozin Treatment of Cats with Hypertension

Bexagliflozin has substantial hypotensive effects in cats. Cats with hypertension managed by bexagliflozin will typically lose a small amount of weight which will be beneficial if they are overweight or obese—as is often the case for hypertensive cats—but will otherwise not be detrimental to the health of cats so managed. Cats with hypertensive diabetes will often gain weight when managed by bexagliflozin because the wasting effect of advanced diabetic disease will be counteracted by the action of bexagliflozin. Cats with hypertension managed with bexagliflozin will need to have adequate water provided to accommodate their increased urine output. A supplementation of the diet to accommodate the increased energy loss by glucosuria may be necessary. If diarrhea or loose stools are observed, the dose can be reduced or the diet changed to provide a decreased fraction of metabolizable energy in the form of carbohydrates. Cats will tolerate bexagliflozin well and owners will express satisfaction with the health of their cats compared to owners of cats with hypertension not managed by bexagliflozin. A single dosage strength of 15 mg, for example a tablet containing 15 mg of bexagliflozin, can be administered to a cat without adjustment for body mass. The fixed dosage can also be divided into equal portions for bid dosing if desired.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method for the treatment of heart failure in a companion animal, comprising administering to the companion animal in need thereof a therapeutically effective amount of a compound that inhibits a sodium-dependent glucose transporter (SGLT) or prodrug thereof.

2. A method for the treatment of chronic kidney disease (CKD) in a companion animal, comprising administering to the companion animal in need thereof a therapeutically effective amount of a compound that inhibits a sodium-dependent glucose transporter (SGLT) or a prodrug thereof.

3. A method for the treatment of hypertension in a companion animal, comprising administering to a companion animal in need thereof a therapeutically effective amount of a compound that inhibits a sodium-dependent glucose transporter (SGLT) or a prodrug thereof.

4. The method of any one of claims 1-3, where the companion animal is preselected to not have diabetes.

5. The method of any one of claims 1-3, where the companion animal is preselected to have diabetes.

6. The method of any one of claims 1-3, where the companion animal is preselected to not have type 2 diabetes.

7. The method of any one of claims 1-3, where the companion animal is preselected to have type 2 diabetes.

8. The method of any one of claims 1-3, where the companion animal is preselected to be obese.

9. The method of any one of claims 1-3, where the companion animal is preselected to not be obese.

10. The method of any one of claims 1-9, wherein the companion animal is a canine.

11. The method of claim 10, wherein the therapeutically effective amount administered to the canine is a total daily dosage of 10-4,000 μg kg⁻¹ of the compound that inhibits an SGLT or the prodrug thereof.

12. The method of claim 10, wherein the therapeutically effective amount administered to the canine is a total daily dosage of 50-3,200 μg kg⁻¹ of the compound that inhibits an SGLT or the prodrug thereof.

13. The method of claim 10, wherein the therapeutically effective amount administered to the canine is a total daily dosage selected from the group consisting of 50 μg μkg⁻¹, 100 μg kg⁻¹, 200 μg kg⁻¹, 400 μg kg⁻¹, 800 μg kg⁻¹, 1,000 μg kg⁻¹, 1,600 μg kg⁻¹, and 3,200 μg kg⁻¹ of the compound that inhibits an SGLT or the prodrug thereof.

14. The method of claim 10, wherein the therapeutically effective amount administered to the canine is a total daily dosage of 1,000 μg kg⁻¹ of the compound that inhibits an SGLT or the prodrug thereof.

15. The method of any one of claims 1 or 4-14, wherein the companion animal is a canine with heart failure.

16. The method of claim 15, wherein, the canine with heart failure is diagnosed with mitral valve disease.

17. The method of any one of claims 2 or 4-14, wherein the companion animal is a canine with CKD.

18. The method of claim 17, wherein the canine is preselected to not be hypertensive.

19. The method of claim 17 or claim 18, wherein the canine is preselected to not have heart failure.

20. The method of any one of claims 3-14, wherein the companion animal is a canine with hypertension.

21. The method of any one of claims 1-9, wherein the companion animal is a feline.

22. The method of claim 21, wherein the therapeutically effective amount administered to a feline is a total daily dosage of 100-30,000 μg kg⁻¹ of the compound that inhibits an SGLT or the prodrug thereof.

23. The method of claim 21, wherein the therapeutically effective amount administered to a feline is a total daily dosage of 200-25,600 μg kg⁻¹ of the compound that inhibits an SGLT or the prodrug thereof.

24. The method of claim 21, wherein the therapeutically effective amount administered to a feline is a total daily dosage selected from the group consisting of 200 μg kg⁻¹, 400 μg kg⁻¹, 800 μg kg⁻¹, 1,000 μg kg⁻¹, 1,600 μg kg⁻¹, 2,500 μg kg⁻¹, 3,200 g kg⁻¹, 5,000 μg kg⁻¹, 6,400 μg kg⁻¹, 12,800 μg kg⁻¹, and 25,600 μg kg⁻¹ of the compound that inhibits an SGLT or the prodrug thereof.

25. The method of claim 21, wherein the therapeutically effective amount administered to a feline is a total daily dosage of 2,500 μg kg⁻¹ or 5,000 μg kg⁻¹ of the compound that inhibits an SGLT.

26. The method of any one of claims 1, 4-9 or 21-25, wherein the companion animal is a feline with heart failure.

27. The method of claim 26, wherein, the feline with heart failure is diagnosed with hypertrophic cardiomyopathy.

28. The method of any one of claims 1, 4-9 or 21-25, wherein the companion animal is a feline with CKD.

29. The method of claim 28, wherein the feline is preselected to not be hypertensive.

30. The method of claim 28 or claim 29, wherein the feline is preselected to not have heart failure.

31. The method of any one of claims 3-9 or 21-25, wherein the companion animal is a feline with hypertension.

32. The method of any one of claims 1-31, wherein the therapeutically effective amount is administered one time, daily.

33. The method of any one of claims 1-31, wherein the therapeutically effective amount is administered twice, daily.

34. The method of any one of claims 1-31, wherein the therapeutically effective amount is administered three times, daily.

35. The method of any one of claims 1-31, wherein the therapeutically effective amount is administered four times, daily.

36. The method of any one of claims 1-35, wherein the therapeutically effective amount is administered as an oral liquid dosage form.

37. The method of any one of claims 1-36, wherein the compound that inhibits an SGLT selectively inhibits SGLT1.

38. The method of any one of claims 1-36, wherein the compound that inhibits an SGLT selectively inhibits SGLT2.

39. The method of any one of claims 1-38, wherein the compound that inhibits an SGLT or the prodrug thereof has a structure selected from the group consisting of 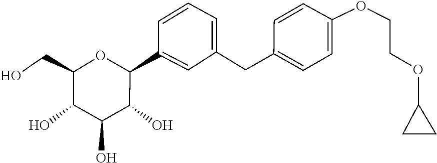 [SMILES: OC[C@H]1O[C@@H](c2cccc(Cc3ccc(OCCOC4CC4)cc3)c2)[C@H](O)[C@@H](O)[C@@H]1O]
(2S,3R,4R,5 S,6R)-2-(4-chloro-3-(4-(2-cyclopropoxyethoxy)benzyl)phenyl)-6-(hydroxymethyl)tetrahydro-2H-pyran-3,4,5-triol 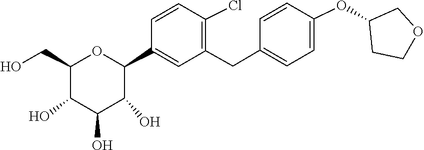 [SMILES: OC[C@H]1O[C@@H](c2ccc(Cl)c(Cc3ccc(O[C@H]4CCOC4)cc3)c2)[C@H](O)[C@@H](O)[C@@H]1O]
(2S,3R,4R,5 S,6R)-2-(4-chloro-3-(4-((S)-tetrahydrofuran-3-yloxy)benzyl)phenyl)-6-(hydroxymethyl)tetrahydro-2H-pyran-3,4,5-triol 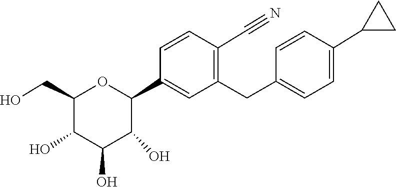 [SMILES: N#Cc1ccc([C@@H]2O[C@H](CO)[C@@H](O)[C@H](O)[C@H]2O)cc1Cc1ccc(C2CC2)cc1]
2-(4-cyclopropylbenzyl)-4-((2S,3R,4R,5S,6R)-3,4,5-trihydroxy-6-(hydroxymethyl)tetrahydro-2H-pyran-2-yl)benzonitrile 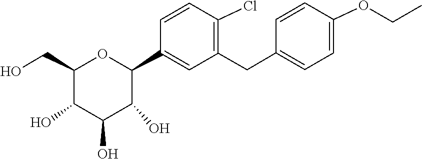 [SMILES: CCOc1ccc(Cc2cc([C@@H]3O[C@H](CO)[C@@H](O)[C@H](O)[C@H]3O)ccc2Cl)cc1]
(2S,3R,4R,5S,6R)-2-(4-chloro-3-(4-ethoxybenzyl)phenyl)-6-(hydroxymethyl)tetrahydro-2H-pyran-3,4,5-triol 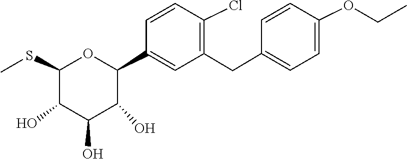 [SMILES: CCOc1ccc(Cc2cc([C@@H]3O[C@H](SC)[C@@H](O)[C@H](O)[C@H]3O)ccc2Cl)cc1]
(2S,3R,4R,5S,6R)-2-(4-chloro-3-(4-ethoxybenzyl)phenyl)-6-(methylthio)tetrahydro-2H-pyran-3,4,5-triol 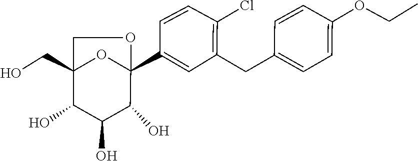 [SMILES: CCOc1ccc(Cc2cc([C@]34OC[C@](CO)(O3)[C@@H](O)[C@H](O)[C@H]4O)ccc2Cl)cc1]
(1S,2S,3S,4R,5S)-5-(4-chloro-3-(4-ethoxybenzyl)phenyl)-1-(hydroxymethyl)-6,8-dioxabicyclo[3.2.1]octane-2,3,4-triol 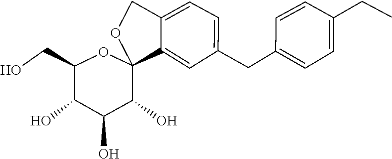 [SMILES: CCc1ccc(Cc2ccc3c(c2)[C@]2(OC3)O[C@H](CO)[C@@H](O)[C@H](O)[C@H]2O)cc1]
(1 S,3′R,4'S,5'S,6′R)-6-(4-ethylbenzyl)-6′-(hydroxymethyl)-3′,4′,5′,6′-tetrahydro-3H-spiro[isobenzofuran-1,2′-pyran]-3′,4′,5-triol 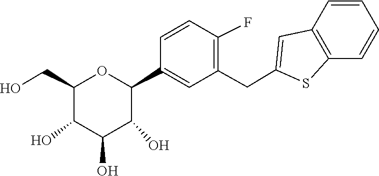 [SMILES: OC[C@H]1O[C@@H](c2ccc(F)c(Cc3cc4ccccc4s3)c2)[C@H](O)[C@@H](O)[C@@H]1O]
(2S,3R,4R,5S,6R)-2-(3-(benzo[b]thiophen-2-ylmethyl)-4-fluorophenyl)-6-(hydroxymethyl)tetrahydro-2H-pyran-3,4,5-triol 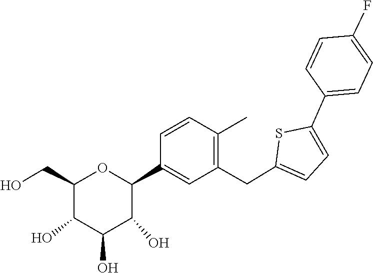 [SMILES: Cc1ccc([C@@H]2O[C@H](CO)[C@@H](O)[C@H](O)[C@H]2O)cc1Cc1ccc(-c2ccc(F)cc2)s1]
(2 S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol 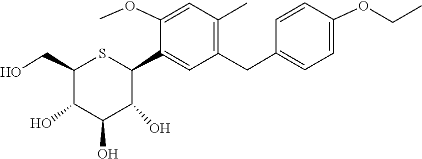 [SMILES: CCOc1ccc(Cc2cc([C@@H]3S[C@H](CO)[C@@H](O)[C@H](O)[C@H]3O)c(OC)cc2C)cc1]
(2S,3R,4R,5S,6R)-2-(5-(4-ethoxybenzyl)-2-methoxy-4-methylphenyl)-6-(hydroxymethyl)tetrahydro-2H-thiopyran-3,4,5-triol 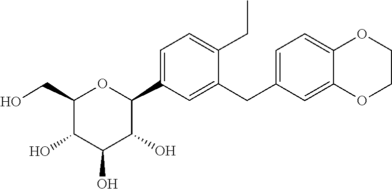 [SMILES: CCc1ccc([C@@H]2O[C@H](CO)[C@@H](O)[C@H](O)[C@H]2O)cc1Cc1ccc2c(c1)OCCO2]
(2S,3R,4R,5S,6R)-2-[3-(2,3-Dihydro-benzo[1,4]dioxin-6-ylmethyl)-4-ethyl-phenyl]-6-hydroxymethyl-tetrahydro-pyran-3,4,5-triol 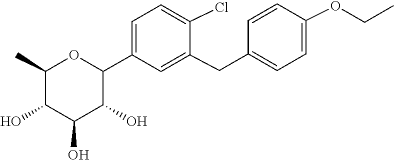 [SMILES: CCOc1ccc(Cc2cc(C3O[C@H](C)[C@@H](O)[C@H](O)[C@H]3O)ccc2Cl)cc1]
(3R,4S,5S,6R)-2-(4-chloro-3-(4-ethoxybenzyl)phenyl)-6-methyltetrahydro-2H-pyran-3,4,5-triol 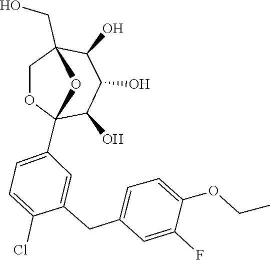 [SMILES: CCOc1ccc(Cc2cc([C@@]34OC[C@@](CO)(O3)[C@@H](O)[C@H](O)[C@H]4O)ccc2Cl)cc1F]
(1R,2S,3S,4R,5R)-5-[4-chloro-3-[(4-ethoxy-3-fluorophenyl)methyl]phenyl]-1-(hydroxymethyl)-6,8-dioxabicyclo[3.2.1]octane-2,3,4-triol 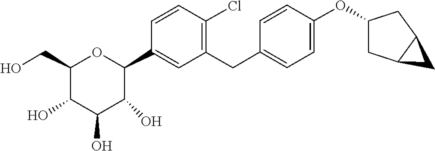 [SMILES: OC[C@H]1O[C@@H](c2ccc(Cl)c(Cc3ccc(O[C@H]4C[C@@H]5C[C@@H]5C4)cc3)c2)[C@H](O)[C@@H](O)[C@@H]1O]
(2S,3R,4R,5S,6R)-2-(3-(4-(((1R,3s,5S)-bicyclo[3.1.0]hexan-3-yl)oxy)benzyl)-4-chlorophenyl)-6-(hydroxymethyl)tetrahydro-2H-pyran-3,4,5-triol 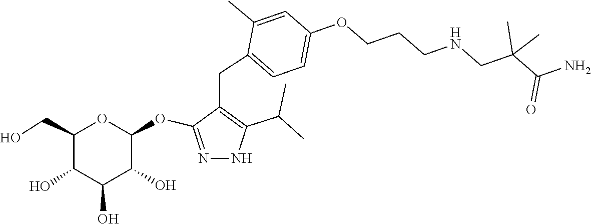 [SMILES: Cc1cc(OCCCNCC(C)(C)C(N)=O)ccc1Cc1c(O[C@@H]2O[C@H](CO)[C@@H](O)[C@H](O)[C@H]2O)n[nH]c1C(C)C]
3-((3-(4-((5-isopropyl-3-(((2S,3R,4S,5S,6R)-3,4,5-trihydroxy-6-(hydroxymethyl)tetrahydro-2H-pyran-2-yl)oxy)-1H-pyrazol-4-yl)methyl)-3-methylphenoxy)propyl)amino)-2,2-dimethylpropanamide 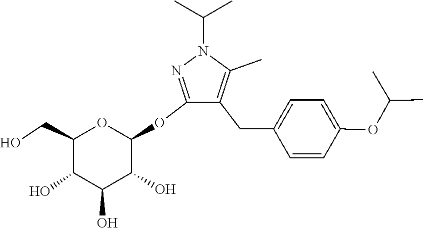 [SMILES: Cc1c(Cc2ccc(OC(C)C)cc2)c(O[C@@H]2O[C@H](CO)[C@@H](O)[C@H](O)[C@H]2O)nn1C(C)C]
(2R,3S,4S,5R,6S)-2-(hydroxymethyl)-6-((4-(4-isopropoxybenzyl)-1-isopropyl-5-methyl-1H-pyrazol-3-yl)oxy)tetrahydro-2H-pyran-3,4,5-triol 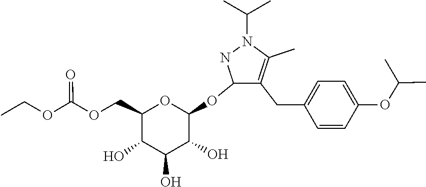
ethyl (((2R,3S,4S,5R,6S)-3,4,5-trihydroxy-6-((4-(4-isopropoxybenzyl)-1-isopropyl-5-methyl-1H-pyrazol-3-yl)oxy)tetrahydro-2H-pyran-2-yl)methyl) carbonate 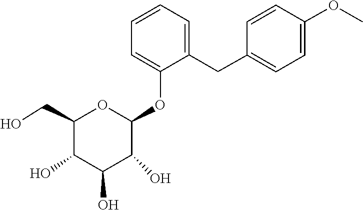 [SMILES: COc1ccc(Cc2ccccc2O[C@@H]2O[C@H](CO)[C@@H](O)[C@H](O)[C@H]2O)cc1]
(2R,3S,4S,5R,6S)-2-(hydroxymethyl)-6-(2-(4-methoxybenzyl)phenoxy)tetrahydro-2H-pyran-3,4,5-triol 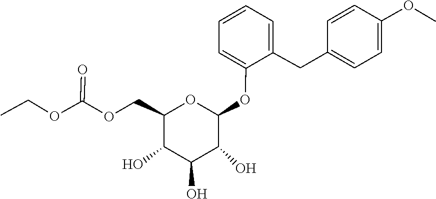 [SMILES: CCOC(=O)OC[C@H]1O[C@@H](Oc2ccccc2Cc2ccc(OC)cc2)[C@H](O)[C@@H](O)[C@@H]1O]
ethyl (((2R,3S,4S,5R,6S)-3,4,5-trihydroxy-6-(2-(4-methoxybenzyl)phenoxy)tetrahydro-2H-pyran-2-yl)methyl) carbonate 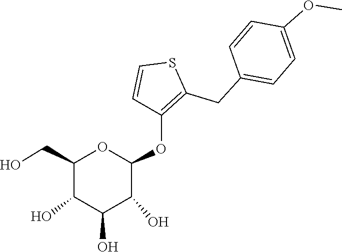 [SMILES: COc1ccc(Cc2sccc2O[C@@H]2O[C@H](CO)[C@@H](O)[C@H](O)[C@H]2O)cc1]
(2R,3 S,4 S,5R,6 S)-2-(hydroxymethyl)-6-(2-(4-methoxybenzyl)thiophen-3-yloxy)tetrahydro-2H-pyran-3,4,5-triol and 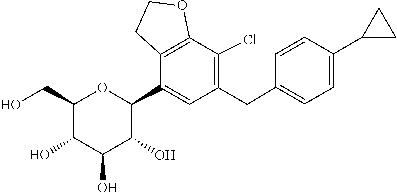 [SMILES: OC[C@H]1O[C@@H](c2cc(Cc3ccc(C4CC4)cc3)c(Cl)c3c2CCO3)[C@H](O)[C@@H](O)[C@@H]1O]
(2S,3R,4R,5S,6R)-2-(7-chloro-6-(4-cyclopropylbenzyl)-2,3-dihydrobenzofuran-4-yl)-6-(hydroxymethyl)tetrahydro-2H-pyran-3,4,5-triol.

40. The method of any one of claims 1-38, wherein the compound that inhibits an SGLT has the structure 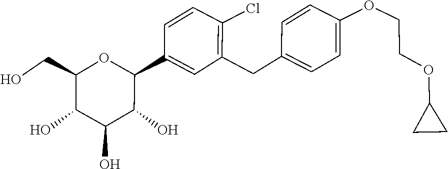 [SMILES: OC[C@H]1O[C@@H](c2ccc(Cl)c(Cc3ccc(OCCOC4CC4)cc3)c2)[C@H](O)[C@@H](O)[C@@H]1O]
(2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol).
